(19) United States
(12) Patent Application Publication
(10) Pub. No.: US 2024/0076406 A1
(43) Pub. Date: Mar. 7, 2024

(54) SINGLE DOMAIN ANTIBODIES TARGETING CA-IX AS WELL AS COMPOSITIONS COMPRISING SAME

(71) Applicant: National Research Council of Canada, Ottawa (CA)

(72) Inventors: Anne EG LENFERINK, Lorraine (CA); Mehdi ARBABI GHAHROUDI, Ottawa (CA); Nazanin ROHANI LARIJANI, Montreal (CA); Scott MCCOMB, Ottawa (CA)

(73) Assignee: National Research Council of Canada, Ottawa, ON (CA)

(21) Appl. No.: 18/262,392

(22) PCT Filed: Jan. 21, 2022
(86) PCT No.: PCT/IB2022/050554

§ 371 Date: Jul. 21, 2023

Related U.S. Application Data
(60) Provisional application No. 63/141,123, filed on Jan. 25, 2021.

(51) Int. Cl. C07K 16/40; A61P 35/00; C12N 15/63

(52) U.S. Cl. CPC C07K 16/40; A61P 35/00; C12N 15/63; C07K 2317/35; C07K 2317/52; C07K 2317/565; C07K 2317/569; C07K 2317/92

(57) ABSTRACT

The present disclosure relates to a single domain antibody that specifically binds to the epitope in the catalytic domain of carbonic anhydrase IX (CA-IX). The single domain antibody has a dissociation constant (K_{D}) of 1×10⁻⁷ or lower for a monomeric form of human CA-IX and/or a dimeric form of human CA-IX.

### TECHNICAL FIELD

The present disclosure generally relates to the field of antibodies, more specifically antibodies for the diagnosis, treatment and/or imaging for cancer that target carbonic anhydrase IX.

### BACKGROUND OF THE ART

Carbonic anhydrases are zinc metalloproteins involved in the catalysis of an essential condensation physiological reaction: carbon dioxide hydration to bicarbonate and a proton: CO₂+H₂O↔+H₂CO₃↔+H⁺+HCO₃⁻. Carbonic anhydrase IX (CA-IX) belongs to the family of carbonic anhydrases (CAs; EC 4.2.1.1) with 15 isoforms known in human. These enzymes are of clinical relevance for the development of anti-cancer therapies. Specifically, two cell surface expressed CA isoforms, namely CA-IX (almost exclusively associated with tumors) and CAXII (overexpressed in some tumor types) are involved in tumorigenesis. CA-IX is a hypoxia-induced extracellular enzyme that is functional in several biological processes necessary for cancer growth and metastasis. This includes pH regulation and cell survival, migration and invasion, maintenance of cancer stem cell (CSC) function, development of the pre-metastatic niche and acquisition of chemo- and radio-resistance (Chafe et al. 2019; McDonald et al. 2012; McDonald and Dedhar 2014; McDonald, Chafe, and Dedhar 2016; Zatovicova et al. 2010). Importantly, CA-IX's contribution to these processes is primarily controlled by its extracellular catalytic activity and the production of protons (H⁺). This, in part, drives the occurrence of acidosis in the extracellular microenvironment and facilitates local invasion through disruption of the extracellular matrix and activation of metalloproteases (Csaderova et al. 2013; Estrella et al. 2013; Swayampakula et al. 2017).

The catalytic domain of the CA family members share a high degree of homology which has presented a challenge in the generation of CA-IX-specific inhibitors. Among the CA inhibitors small molecule inhibitor (SMIs) targeting CA-IX have shown most promise. These include the ureidosulfonamides, glycosyl coumarins, and indanesulfonamides which have been shown to inhibit tumor growth in preclinical models of hypoxic, CA-IX-positive breast and colorectal cancer. To increase specificity of CA-IX specific SMIs, several strategies have evolved exploiting the difference in the active site residues, such as the addition of charge, or bulky entities like albumin or hydrophilic sugar moieties. These strategies have improved the selectivity of pan-CA inhibitors, and has led to generation of a series of novel, potent, CA-IX-selective “next generation” SMIs, such as SLC-0111, which has shown an improved affinity for CA-IX's catalytic active site compared to more ubiquitously expressed CA-I and CA-II.

Due to limitations in the specificity to CA-IX and off-target toxicity challenges attributed to the small molecules, recent efforts have focused on identifying novel anti-CA-IX antibodies mAbs, the most prominent of which is cG250 mAb, which interacts with CA-IX's catalytic domain without inhibiting its enzyme activity. Indeed, the disadvantage of small molecules (lack of specificity) leads to a higher likelihood for deleterious secondary effects. Studies using cG250 indicated that this mAb induces antibody-dependent cellular cytotoxicity (ADCC) response. However its use as a naked antibody has shown no significant benefit to disease-free survival rate of patients (>6-year span) in clinical trials, carried out by WILEX AG, compared to a placebo. The company announced however that their late-stage trial, referred to as the ARISER study, did not meet its primary endpoint of median disease-free survival and further development of ‘naked’ cG250 as an anti-cancer treatment was therefore terminated. cG250 is currently used as an imaging diagnostic agent for the detection of clear cell renal carcinoma, for example, Telix pharmaceuticals is using cG250 as an imaging tracer and potential Radio-Immuno Conjugate.

cG250, together with other anti-CA-IX mAbs, have been further pursued with the goal to deliver cytotoxic agents (i.e Antibody-Drug Conjugate, ADC) or radionuclides (i.e Radio-Immuno Conjugate, RIT) into CA-IX expressing tumor cells. For example the 3ee9 Fab was isolated by panning recombinant human CA-IX extracellular domain (ECD) against a library of human Fabs. This Fab engineered into a mAb was further developed as an ADC by BAYER Healthcare BAY79-4620 by conjugation to monomethyl auristatin E (MMAE). BAY79-4620 showed potent antitumor efficacy and a Phase I clinical trial to determine the maximal tolerated dose (MTD) was recently completed by BAYER Healthcare (NCT01028755 and NCT01065623). Another study reports the use of VII/20 (hybridoma-derived) mAb (Zatovicova et al. 2010) inhibited enzyme activity in vitro and tumor growth inhibition in vivo using freshly inoculated HT-29 colorectal tumor cells, however only limited effects were reported on established tumors. Overall there are only few publications that describe immunotherapy based approaches targeting CA-IX or improvements in the development of these CA-IX targeting moieties for cancer imaging, diagnosis and/or treatment.

### SUMMARY

In one aspect there is provided a single domain antibody specifically binding to an epitope in a catalytic domain of carbonic anhydrase IX (CA-IX), wherein the single domain antibody has a dissociation constant (K_{D}) of 1×10⁻⁷ or lower for a monomeric form of CA-IX and/or a dimeric form of CA-IX.

In one embodiment, the single domain antibody has a K_{D} of 9×10⁻⁹ or lower for a CA-IX fragment lacking a proteoglycan-like (PG) domain.

In one embodiment, the single domain antibody is capable of reducing the biological activity of CA-IX.

In one embodiment, the single domain antibody is capable of being internalized by a cell expressing CA-IX.

In one embodiment, the epitope is a conformational epitope or a linear epitope.

In one embodiment, the single domain antibody lacks the ability to specifically bind to at least one of carbonic anhydrase II (CA-II), carbonic anhydrase IV (CA-IV), carbonic anhydrase XII (CA-XII), carbonic anhydrase XIV (CA-XIV), and/or a proteoglycan-like (PG) domain of CA-IX.

In one embodiment, the single domain antibody comprises:
a first complementary determining region (CDR) having the amino acid sequence of SEQ ID NO: 2, a variant of SEQ ID NO: 2 or a fragment of SEQ ID NO: 2; SEQ ID NO: 6, a variant of SEQ ID NO: 6 or a fragment of SEQ ID NO: 6; or SEQ ID NO: 10, a variant of SEQ ID NO: 10 or a fragment of SEQ ID NO: 10;
a second CDR having the amino acid sequence of sequence of SEQ ID NO: 3, a variant of SEQ ID NO: 3 or a fragment of SEQ ID NO: 3; SEQ ID NO: 7, a variant of SEQ ID NO: 7 or a fragment of SEQ ID NO: 7; or SEQ ID NO: 11, a variant of SEQ ID NO: 11 or a fragment of SEQ ID NO: 11; and/or
a third CDR having the amino acid sequence of SEQ ID NO: 4, a variant of SEQ ID NO: 4 or a fragment of SEQ ID NO: 4; SEQ ID NO: 8, a variant of SEQ ID NO: 8 or a fragment of SEQ ID NO: 8; or SEQ ID NO: 12, a variant of SEQ ID NO: 12 or a fragment of SEQ ID NO: 12.

In one embodiment, the single domain antibody comprises at least one of the first CDR having the amino acid sequence of SEQ ID NO: 2, a variant of SEQ ID NO: 2 or a fragment of SEQ ID NO: 2; the second CDR having the amino acid sequence of SEQ ID NO: 3, a variant of SEQ ID NO: 3 or a fragment of SEQ ID NO: 3 or the third CDR having the amino acid sequence of SEQ ID NO: 4, a variant of SEQ ID NO: 4 or a fragment of SEQ ID NO: 4.

In one embodiment, the single domain antibody has the amino acid sequence of SEQ ID NO: 1, a variant of SEQ ID NO: 1 or a fragment of SEQ ID NO: 1.

In one embodiment, the single domain antibody comprises at least one of the first CDR having the amino acid sequence of SEQ ID NO: 6, a variant of SEQ ID NO: 6 or a fragment of SEQ ID NO: 6; the second CDR having the amino acid sequence of SEQ ID NO: 7, a variant of SEQ ID NO: 7 or a fragment of SEQ ID NO: 7; or the third CDR having the amino acid sequence of SEQ ID NO: 8, a variant of SEQ ID NO: 8 or a fragment of SEQ ID NO: 8.

In one embodiment, the single domain antibody has the amino acid sequence of SEQ ID NO: 5, a variant of SEQ ID NO: 5 or a fragment of SEQ ID NO: 5, a variant of SEQ ID NO: 5 or a fragment of SEQ ID NO: 5.

In one embodiment, the single domain antibody comprises at least one of the first CDR having the amino acid sequence of SEQ ID NO: 10, a variant of SEQ ID NO: 10 or a fragment of SEQ ID NO: 10; the second CDR having the amino acid sequence of SEQ ID NO: 11, a variant of SEQ ID NO: 11 or a fragment of SEQ ID NO: 11; or the third CDR having the amino acid sequence of SEQ ID NO: 12, a variant of SEQ ID NO: 12 or a fragment of SEQ ID NO: 12.

In one embodiment, the single domain antibody has the amino acid sequence of SEQ ID NO: 9, a variant of SEQ ID NO: 9 or a fragment of SEQ ID NO: 9.

In one embodiment, the single domain antibody is a V_{H}H antibody.

In one embodiment, the single domain antibody is a nanobody.

In one embodiment, the single domain antibody of is associated with a toxic payload or a detectable label.

In one aspect there is provided a multivalent antibody comprising at least one of the single domain antibody according to the present disclosure.

In one embodiment, the multivalent antibody comprises the at least one single domain antibody capable of reducing the biological activity of CA-IX.

In one aspect there is provided a chimeric polypeptide comprising (i) at least one the single domain antibody or the multivalent antibody and (ii) a carrier polypeptide.

In one embodiment, the carrier polypeptide is a Fc and can have, for example, the amino acid sequence of SEQ ID NO: 15, is a variant of the amino acid sequence of SEQ ID NO: 15 or is a fragment of the amino acid sequence of SEQ ID NO: 15.

In one embodiment, the chimeric polypeptide comprises the at least one single domain antibody capable of reducing the biological activity of CA-IX.

In one embodiment, the chimeric polypeptide further comprises a linker between the at least one single domain antibody or the multivalent antibody and the carrier polypeptide. The linker can have, in some embodiments, the amino acid sequence of SEQ ID NO: 14, is a variant of the amino acid sequence of SEQ ID NO: 14 or is a variant of the amino acid sequence of SEQ ID NO: 14.

In one embodiment, the chimeric polypeptide can have the amino acid sequence of SEQ ID NO: 16, 17 or 18, be a variant of the amino acid sequence of SEQ ID NO: 16, 17 or 18 or be a fragment of the amino acid sequence of SEQ ID NO: 16, 17 or 18.

In one embodiment, the carrier polypeptide is an antibody, an antibody fragment, a serum protein, a chimeric antigen receptor (CAR) construct or a bispecific T cell engager (BiTE) construct.

In one embodiment, the chimeric polypeptide is associated with a toxic payload or a detectable label.

In one aspect, there is provided a nucleic acid molecule encoding the single domain antibody, the multivalent antibody or the chimeric polypeptide.

In one embodiment, the nucleic acid molecule comprises at least one of SEQ ID NO: 21, 22, 23, 24, 25 or 26, a variant thereof or a fragment thereof.

In one aspect, there is provided a vector comprising the nucleic acid molecule described herein.

In one aspect there is provided a recombinant host cell comprising the nucleic acid molecule described herein or the vector described herein.

In one aspect there is provided a method of limiting the biological activity of carbonic anhydrase IX (CA-IX) expressed by a cell, the method comprising contacting the single domain antibody, the multivalent antibody or the chimeric polypeptide with CA-IX expressed by the cell so as to limit the biological activity of CA-IX in the cell when compared to a control cell contacted by a control single domain antibody that fails to specifically bind to the catalytic domain of CA-IX and reduce the biological activity of CA-IX.

In one embodiment, the method for the alleviation of a symptom of a cancer, the treatment of a cancer, the prevention of the re-occurrence of cancer, delivery of a toxic payload to the cell, and/or for improving the usefulness of a further therapeutic agent.

In one aspect, there is provided a method of detecting a cell expressing or overexpressing carbonic anhydrase IX (CA-IX), the method comprising:
contacting the single domain antibody, the multivalent antibody or the chimeric polypeptide with the cell under conditions so as to allow the specific binding of the single domain antibody, the multivalent antibody or the chimeric polypeptide to the cell; and
determining the presence of a complex formed between the cell and the single domain antibody or the chimeric polypeptide; and
detecting the cell as expressing or overexpressing CA-IX if the presence of the complex is determined to be present.

In one embodiment, the cell is a cancerous cell, an hypoxic cell and/or a pre-necrotic cell.

In one embodiment, the cell is present in a subject.

In one aspect, there is provided the use of the single domain antibody, the multivalent antibody or the chimeric polypeptide for the treatment of cancer in a subject in need thereof.

In one aspect, there is provided the single domain antibody, the multivalent antibody or the chimeric polypeptide for use in the treatment of cancer in a subject in need thereof.

In one aspect, there is provided the single domain antibody, the multivalent antibody or the chimeric polypeptide for use in the manufacture of a medicament for the treatment of cancer in a subject in need thereof.

In one aspect, there is provided the use of the single domain antibody, the multivalent antibody or the chimeric polypeptide in the manufacture of a medicament for the treatment of cancer in a subject in need thereof.

Many further features and combinations thereof concerning the present improvements will appear to those skilled in the art following a reading of the instant disclosure.

### DESCRIPTION OF THE DRAWINGS

FIG. 1 is a schematic of the human CA-IX (hCA-IX) enzyme in one binding conformation.

FIG. 2 is a schematic of the hCA-IX enzyme in another binding conformation.

FIG. 3A is a graph showing the binding kinetics determined by surface plasmon resonance (SPR) for 3 CA-IX single domain antibodies (sdCA9-1, -2, -3-V_{H}H) to the dimeric extracellular domain (ECD) of hCA-IX.

FIG. 3B shows graph of bio-layered interferometry (BLI) Octet measurements showing the binding magnitude of wild type (WT) hCA-IX ECD monomer and mutated monomeric hCA-IX ECD (lacking the PG domain; APG-hCA-IX-ECD) for Fc-captured sdCA9-1 and -2-Fc fused antibodies.

FIG. 4 is a schematic of a cellular CA-IX binding to a single domain antibody adapted from Nocentini et al., 2018. The drawing shows the intracellular/extracellular interface 6 for CA-IX 7. CA-IX 7 is shown in its dimeric form have a first dimer 7a and a second dimer 7b, the PG domain 8, and crosses the lipid bilayer 9 of the cell. The sdCA9-2-Fc 2 is shown binding to the catalytic domain of CA-IX 7.

FIG. 5 shows graphs of the binding affinity measurements of Fc-fused sdCA9 antibodies using human clear cell renal cell carcinoma SKRC52 (hCA-IX high, left panel) and SKRC59 (hCA-IX low, right panel) expressing cells. Cell binding assays were performed using a plate-based format, binding was expressed as the mean fluorescent intensity per cell area. (control IgG (●), cG250 (▪), sdCA9-1-Fc (▴), and sdCA9-2-Fc (▾)). K_{D} values were determined by plotting fluorescent intensity measured for each condition of the sdCA9-Fc antibodies using Graphpad Prism. cG250 full sized antibody (FSA) and isotype matching IgG FSA were used as bench mark and negative control, respectively.

FIG. 6 shows graphs of the isoform and species cross reactivity measurement for sdCA9-V_{H}H antibodies. Binding above background was measured in an ELISA assay for the binding of 500 nM of sdCA9-V_{H}H antibodies to human CA-II, CA-IV, CA-IX, CA-XII and CA-XIV (isoform cross reactivity, upper panel), then CA-IX for mouse, human, cynomolgus monkey and dog (species cross reactivity, lower panel). (sdCA9-1-V_{H}H ( [symbol] ) and sdCA9-2-V_{H}H ( [symbol] )

FIG. 7 shows a graph of hCA-IX in vitro enzyme activity assay. Dose-dependent inhibition of hCA-IX catalytic activity in vitro through measuring 4-methylumbelliferyl acetate (4Mu-Ac) substrate hydrolysis by purified hCA-IX dimer protein for sdCA9-1-V_{H}H and sdCA9-2-V_{H}H compared to hCA-IX/XII SMI U104 (AKA SLC-0111) and full sized antibody (FSA) m4A2. sdCA9-2-V_{H}H inhibited the hCA-IX enzyme activity to similar levels achieved by SMI U104 (AKA SLC-0111). (U104 (●), sdCA9-1-V_{H}H (▪), sdCA9-2-V_{H}H (▴), and m4A2 (▾)).

FIG. 8 shows a graph of the internalization of the two Fc fused-single domain sdCA9-1-Fc, sdCA9-2-Fc, antibodies upon binding to hCA-IX negative parental mouse mammary carcinoma 67NR cells and hCA-IX overexpressing 67NR (67NR^{hCA-IX}) cells. The increase in fluorescence intensity of the pH sensitive FabFluor-conjugated sdCA9-1-Fc, sdCA9-2-Fc internalization was quantified over time by measuring the fluorescent area for each time point normalized to cell area. An isotype matching hIgG was used as control. (67NR: sdCA9-1-Fc ( [symbol] ), sdCA9-1-Fc ( [symbol] ), hIgG ( [symbol] ); and 67NR^{hCA-IX}: sdCA9-1-Fc (▾), sdCA9-2-Fc (+), and hIgG FSA (●)).

FIG. 9 shows the effect of antibody treatment (sdCA9-1-V_{H}H (▪), sdCA9-2-V_{H}H (▴); 100 μg/mL) over time on the growth of mouse mammary carcinoma hCA-IX negative parental 67NR (top left panel) and hCA-IX overexpressing 67NR cells (67NR^{hCA-IX}, top right panel), compared to non-treated control spheroid (control (●)). This data shows that sdCA9-2-V_{H}H specifically inhibits the growth of 67NR^{hCA-IX} spheroids over time similar to SMI U104 (SLC-0111) (▾). Bottom panels show a dose dependent effect of sdCA9-1-V_{H}H and sdCA9-2-V_{H}H relative to SMI U104 (control ( [symbol] ), sdCA9-1-V_{H}H ( [symbol] ), sdCA9-2-V_{H}H ( [symbol] ) and U104 (SLC0111) ( [symbol] ), lower panels) in the parental 67NR cells (left) and 67NR^{hCA-IX} cells. sdCA9-1-V_{H}H exerted a hCA-IX non-specific phenotype that can also be observed in the hCA-IX negative parental 67NR cell line.

FIG. 10 The molecular structure of a CAIX-specific single domain antibody bi-specific T cell engager proteins with a hinge/spacer domain; a sdCA9-V_{H}H sequence at the 5′ end of a DNA construct, followed by a linker sequence which can be of varying composition, followed by a CD3-specific single chain variable fragment.

FIG. 11A depicts the results of Jurkat cell bi-specific T cell engager activity assay wherein different dilutions of HEK293T supernatants containing CAIX bi-specific T cell engager molecules (CAIX-2-1E2 BITE) was placed on top of co-cultures containing Jurkat cells along with either CAIX negative human B lymphocyte cell line (Raji cells, left panel) or human clear cell renal cell carcinoma SKRC52 (hCA-IX high, right panel) target cells. Graphs depict the average CD69-specific antibody staining of Jurkat cells as measured by flow cytometry. Error bars present the standard error of the mean over 2 duplicate co-culture wells. Results demonstrate CAIX-antigen specific activation of Jurkat (T cells) in the presence of novel CAIX-sdAb bi-specific T cell engager molecules.

FIG. 11B shows the results of Jurkat cell activation measured by reading number of activated Jurkat cells using fluorescent signal from CD69 expression on activated Jurkats, CAIX-2-1E2-BiTE supernatant were deposited on co-cultures containing Jurkat CD69-td tomato reporter cells along with SKRC52(CAIX-positive) or SKRC59 (CAIX-negative) target cells grown either in 2D monolayer cultures (top panel) or in 3D (bottom panel) as tumor spheroids. Number of activated Jurkats are reported by counting the number of positive td-tomato expressing cells (images on the left). Error bars present duplicate experiments and mean count of positive Jurkat cells per image from 4 wells. The results demonstrate dose dependent, and CAIX specific activation of Jurkat (T cells) in the presence varying doses of sdCAIX-2 bi specific CD3 T cell engager antibodies.

FIG. 12 depicts the molecular structure of a single-binder CAIX-specific chimeric antigen receptor; wherein a sdCA9-V_{H}H sequence at the 5′ end of a CAR DNA construct is followed by a linker sequence which can be of varying composition, followed by a similar structure to other CAR molecules [hinge domain, transmembrane domain, intracellular signaling domain(s)].

FIG. 13 Depicts the results of CAR-Jurkat activity assay wherein Jurkat cells were transiently electroporated with sdCA9-1-V_{H}H, sdCA9-2-V_{H}H CAR or an irrelevant target CD19 CAR plasmid as a control and cultured either alone or in co-culture with CAIX-positive (SKRC52 cell line) or CAIX-negative (SKOV3) cell lines. The level of T cell activation was measured using human CD69-specific antibody staining and flow cytometry. Graphs depict the mean fluorescent intensity for CD69-staining for each single domain antibody targeted CAR constructs performed in a single experiment in duplicate, either in culture with no target cells (first bar), CAIX negative SKOV3 target cells (second bar), or CAIX positive SKRC52 target cells (third bar). Error bars show the standard error of the mean for duplicate wells. Results demonstrate CAIX-specific response with the two novel sdCA9-1 and 2 V_{H}H CAR constructs tested.

### DETAILED DESCRIPTION

### Abbreviations

CA=carbonic anhydrase
hCA=human carbonic anhydrase
Ig=immunoglubulin
K_{D}=dissociation constant
sdAb=single domain antibody
V_{H}H=variable region of the heavy chain of a camelid antibody
Fᵥ=antigen binding region of antibody
F_{c}=constant region of antibody
Fc-fused=single domain antibody fused to a Fragment crystallizable (Fc) region of an immunoglubulin (Ig)
V_{L}=immunoglobulin variable light chain
V_{H}=immunoglobulin variable heavy chain

### Definitions

The term “antibody”, also referred to in the art as “immunoglobulin”, as used herein refers to a protein comprising at least one heavy or light polypeptide chain. In an embodiment, an antibody comprises a paired heavy and light polypeptide chains. In humans, various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable V_{L} and a constant domain, while the heavy chain folds into a variable V_{H} and three constant domains. Interaction of the heavy and light chain variable domains results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important biochemical events. The variable region of an antibody contains the antigen-binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape, and chemistry of the surface they present to the antigen. Various schemes exist for identification of the regions of hypervariability, the two most common being those of Kabat and of Chothia and Lesk. Kabat et al (1991) define the “complementarity-determining regions” (CDR) based on sequence variability at the antigen-binding regions of the V_{H} and V_{L} domains. Chothia and Lesk (1987) define the “hypervariable loops” (H or L) based on the location of the structural loop regions in the V_{H} and V_{L} domains. As these individual schemes define CDR and hypervariable loop regions that are adjacent or overlapping, those of skill in the antibody art often utilize the terms “CDR” and “hypervariable loop” interchangeably, and they may be so used herein. The CDR/loops are referred to herein according to the more recent IMGT numbering system (Lefranc, M.-P. et al., 2003), which was developed to facilitate comparison of variable domains. In this system, conserved amino acids (such as Cys23, Trp41, Cys104, Phe/Trp118, and a hydrophobic residue at position 89) always have the same position. Additionally, a standardized delimitation of the framework regions (FR1: positions 1 to 26; FR2: 39 to 55; FR3: 66 to 104; and FR4: 118 to 129) and of the CDR (CDR1: 27 to 38, CDR2: 56 to 65; and CDR3: 105 to 117) is provided.

A “single domain antibody” or “sdAb” as used herein refers to an antibody that has a single monomeric variable antibody domain comprising at least three complementary determining regions (CDRs). As it is known in the art, the single domain antibodies can be obtained from camelids (called also V_{H}H antibodies or nanobodies), from fish (called V_{NAR} antibodies), or by using phage display technology. The single domain antibodies can also be derived from a heavy chain (V_{H}) or a light chain (V_{L}) of an immunoglobulin. Examples of single domain antibodies of the present disclosure include but are not limited to V_{H}H or nanobodies. Single domain antibodies may be further defined by a size of less than about 15 kDa, less than about 14 kDa, less than about 13 kDa, less than about 12 kDa, less than about 11 kDa, or less than about 10 kDa. In one embodiment, sdAbs or nanobodies can be defined as having less than about 150 amino acids, less than about 140 amino acids, less than about 130 amino acids, less than about 120 amino acids, less than about 110 amino acids or less than 100 amino acids. In a further embodiment, nanobodies can be defined as consisting of less than about 150 amino acids, less than about 140 amino acids, less than about 130 amino acids, less than about 120 amino acids, less than about 110 amino acids or less than 100 amino acids. Single domain antibodies, especially camelid antibodies, present many advantages including a high expression yield, a high solubility with little or no aggregation tendency, and due to their size can recognize epitopes not accessible to conventional antibodies and conventional antibody fragments. Without wishing to be bound by theory, the single domain antibodies are able to identify epitopes not recognized by other antibodies due to their protruding CDR 3 loop. In addition to their high affinity, sdAbs are more soluble and stable than other antibody fragments in extreme conditions such as high temperature and pH. sdAbs also have better tissue penetration and are less immunogenic than conventional larger antibodies (such as immunoglobulins). Due to their small size, single domain antibodies generally have a very short serum half-life (due mostly to kidney clearance) compared to full size antibodies. Moreover, the rapid clearance or short circulation half-life, even in conjugated forms, is an advantage for imaging and the treatment of some tumors. In one example, the single domain antibody or the nanobody comprises or consists of an amino acid sequence of SEQ ID NO: 1, 5 or 9, a variant or a fragment thereof. In yet another embodiment, the single domain antibody or the nanobody is encoded by a nucleic acid molecule which comprises or consists of a nucleotide sequence of SEQ ID NO: 21, 22 or 23, a variant (such as a degenerate variant) or a fragment thereof.

The sdAb of the present disclosure may be derived from naturally-occurring sources. Heavy chain antibodies of camelid origin (Hamers-Casterman et al., 1993) lack light chains and thus their antigen binding sites consist of one domain, termed V_{H}H. sdAb have also been observed in shark and are termed V_{NAR} (Nuttall et al., 2003). Other sdAb may be engineered based on human Ig heavy and light chain sequences (Jespers et al., 2004; To et al., 2005). The term “sdAb” may include those sdAb directly isolated from V_{H}H or V_{NAR}, those synthetically prepared from human light or heavy chains, and those obtained from phage display or other technologies. The sdAb can be derived from the aforementioned sdAb, recombinantly produced sdAb, as well as those sdAb generated through affinity maturation, stabilization, solubilization, camelization, or other methods of antibody engineering.

SdAb possess desirable properties for antibody molecules, such as high thermostability, high detergent resistance, relatively high resistance to proteases (Dumoulin et al., 2002) and high production yield (Arbabi-Ghahroudi et al., 1997); they can also be engineered to have very high affinity by isolation from an immune library (Li et al, 2009) or by in vitro affinity maturation (Davies & Riechmann, 1996). Further modifications to increase stability, such as the introduction of non-canonical disulfide bonds (Hussack et al., 2011; Kim et al., 2012), may also be brought to the sdAb.

A person of skill in the art would be well-acquainted with the structure of a single-domain antibody (see, for example, 3DWT, 2P42 in Protein Data Bank). In one example, an sdAb comprises a single immunoglobulin domain that retains the immunoglobulin fold; most notably, only three CDR/hypervariable loops form the antigen-binding site. However, and as would be understood by those of skill in the art, not all CDR may be required for binding the antigen. For example, and without wishing to be limiting, one, two, or three of the CDR may contribute to binding and recognition of the antigen by the sdAb of the present disclosure. The CDR of the sdAb or variable domain are referred to herein as CDR1, CDR2, and CDR3, and numbered as defined by Lefranc, M.-P. et al. (2003).

The sdAb may be of camelid origin or derived from a camelid V_{H}H, and thus may be based on camelid framework regions; alternatively, the CDR described above may be grafted onto V_{NAR}, or V_{H}H. In yet another alternative, the hypervariable loops described above may be grafted onto the framework regions of other types of antibody fragments of any source (for example, mouse) or proteins of similar size and nature onto which CDR can be grafted (for example, see Nicaise et al., 2004).

As used in the context of the present disclosure, a “variant” or a “functional variant” refers to alterations in the amino acid sequence of a protein or a peptide (like a CDR) that do not adversely affect the biological function(s) of the protein or the peptide. In an embodiment, the variant has at least 50%, 55%, 60%, 65%, 70%, 75%, 80%, 85%, 90%, 95%, 96%, 97%, 98% or 99% of the biological activity when compared to the wild-type (unmodified) protein (such as a single domain antibody as described herein, and for example, more specifically, those having the amino acid sequence of SEQ ID NO: 1, 5 or 9) or peptide (for example, a CDR having the amino acid sequence of SEQ ID NO: 2, 3, 4, 6, 7, 8, 10, 11 or 12). In an embodiment, the variant has at least 50%, 55%, 60%, 65%, 70%, 75%, 80%, 85%, 90%, 95%, 96%, 97%, 98% or 99% identity to the wild-type (unmodified) protein (such as a single domain antibody as described herein, and for example, more specifically, those having the amino acid sequence of SEQ ID NO: 1, 5 or 9) or peptide (for example, a CDR having the amino acid sequence of SEQ ID NO: 2, 3, 4, 6, 7, 8, 10, 11 or 12).

A “variant” or a “functional variant” can also refer to alterations in the nucleic acid sequence of a nucleic acid molecule encoding a peptide or a polypeptide of interest. In some embodiments, the variant has at least 50%, 55%, 60%, 65%, 70%, 75%, 80%, 85%, 90%, 95%, 96%, 97%, 98% or 99% identity to the nucleic acid molecule it is based on. In some specific embodiments, a variant of a nucleic acid molecule can correspond to degenerate sequence encoding the same amino acid sequence than the nucleic acid molecule it is based on.

The variant described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

As used in the context of the present disclosure, a “fragment” or a “functional fragment” refers to a reduction of at least one amino acid residue in the amino acid sequence of a protein or a peptide (like a CDR) that do not adversely affect the biological functions of the protein or peptide sequence it is based on. In some embodiments, the fragment has at least 50%, 55%, 60%, 65%, 70%, 75%, 80%, 85%, 90%, 95%, 96%, 97%, 98% or 99% of the biological activity associated with the full-length protein (such as a single domain antibody as described herein, and for example, more specifically, those having the amino acid sequence of SEQ ID NO: 1, 5 or 9 or variants thereof) or peptide (for example, a CDR having the amino acid sequence of SEQ ID NO: 2, 3, 4, 6, 7, 8, 10, 11 or 12 or variants thereof) it is based on. In some embodiments, the fragment has at least 50%, 55%, 60%, 65%, 70%, 75%, 80%, 85%, 90%, 95%, 96%, 97%, 98% or 99% identity to the protein (such as a single domain antibody as described herein, and for example, more specifically, those having the amino acid sequence of SEQ ID NO: 1, 5 or 9 of variants thereof) or peptide (for example, a CDR having the amino acid sequence of SEQ ID NO: 2, 3, 4, 6, 7, 8, 10, 11 or 12 of variants thereof) it is based on.

A “fragment” or a “functional fragment” can also refer to the reduction of at least one nucleic acid residue in the nucleic acid sequence encoding a peptide or a polypeptide of interest. In some embodiments, the fragment has at least 50%, 55%, 60%, 65%, 70%, 75%, 80%, 85%, 90%, 95%, 96%, 97%, 98% or 99% identity to the nucleic acid molecule it is based on. In some specific embodiments, a fragment of a nucleic acid molecule can correspond to a sequence to section encoding the leader sequence has been removed.

The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, BLAST-N, or FASTA-N, or any other appropriate software that is known in the art.

The term “substantially identical sequence” as used herein, may comprise one or more conservative amino acid mutations. It is known in the art that one or more conservative amino acid mutations to a reference sequence may yield a mutant peptide with no substantial change in physiological, chemical, physico-chemical or functional properties compared to the reference sequence; in such a case, the reference and mutant sequences would be considered “substantially identical” polypeptides. In one embodiment, a conservative amino acid substitution is defined herein as the substitution of an amino acid residue for another amino acid residue with similar chemical properties (e.g. size, charge, or polarity). In some embodiments, these conservative amino acid mutations may be made to one or more framework regions of the sdAb while maintaining the CDR regions listed above and the overall structure of the CDR of the antibody or fragment; thus the specificity and binding of the antibody are maintained. In additional embodiments, these conservative amino acid mutations may be made to one or more framework regions and in one or more CDR regions listed above and the overall structure of the CDR of the antibody or fragment; thus the specificity and binding of the antibody are maintained.

In a non-limiting example, a conservative mutation may be an amino acid substitution. Such a conservative amino acid substitution may substitute a basic, neutral, hydrophobic, or acidic amino acid for another of the same chemical group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (lie or 1), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pK value of less than 7, which are typically negatively charged at physiological pH. Acidic amino acids include glutamate (Glu or E), and aspartate (Asp or D).

The substantially identical sequences of the present invention may be at least 90% identical; in another example, the substantially identical sequences may be at least 90, 91, 92, 93, 94, 95, 96, 97, 98, 99, or 100% identical, or any percentage there between, at the amino acid level to sequences described herein. In one embodiment, the substantially identical sequences have less than 10, 9, 8, 7, 6, 5, 4, 3, or 2 conserved substitutions or have one conserved substitution. Importantly, the substantially identical sequences retain the activity and specificity of the reference sequence. In a non-limiting embodiment, the difference in sequence identity may be due to conservative amino acid mutation(s). In a non-limiting example, the present invention may be directed to an antibody or fragment thereof comprising a sequence at least 95%, 98%, or 99% identical to that of the antibodies described herein.

The sdAb of the present disclosure can be provided as a multimer. Multimerization may be achieved by any suitable method of known in the art. For example, and without wishing to be limiting in any manner, multimerization may be achieved using self-assembly molecules such as those described in Zhang et al. (2004a; 2004b) and WO2003/046560, where pentabodies are produced by expressing a fusion protein comprising the antibody or fragment thereof of the present invention and the pentamerization domain of the B-subunit of an AB₅ toxin family (Merritt & Hol, 1995). A multimer may also be formed using the multimerization domains described by Zhu et al. (2010); this form, referred to herein as a “combody” form, is a fusion of the antibody or fragment of the present invention with a coiled-coil peptide resulting in a multimeric molecule (Zhu et al., 2010). Other forms of multivalent display are also encompassed by the present invention. For example, and without wishing to be limiting, the antibody or fragment thereof may be presented as a dimer, a trimer, or any other suitable oligomer. This may be achieved by methods known in the art, for example direct linking connection (Nielson et al., 2000), c-jun/Fos interaction de Kruif & Logtenberg, 1996), “Knob into holes” interaction (Ridgway et al., 1996).

Another method known in the art for multimerization is to dimerize the antibody or fragment thereof using an Fc domain, for example, but not limited to human Fc domains. The Fc domains may be selected from various classes including, but not limited to, IgG, IgM, or various subclasses including, but not limited to IgG1, IgG2, etc. In this approach, the Fc gene in inserted into a vector along with the sdAb gene to generate a sdAb-Fc fusion protein (Bell et al., 2010; Iqbal et al., 2010); the fusion protein is recombinantly expressed then purified. For example, and without wishing to be limiting in any manner, multivalent display formats may encompass chimeric or humanized formats of anti-CA9V_{H}H linked to an Fc domain, or bi- or tri-specific antibody fusions with two or three anti-CA9 V_{H}H recognizing unique epitopes. Such antibodies are easy to engineer and to produce, can greatly extend the serum half-life of sdAb, and may be excellent tumor imaging reagents (Bell et al., 2010).

The Fc domain in the multimeric complex as just described may be any suitable Fc fragment known in the art. The Fc fragment may be from any suitable source; for example, the Fc may be of mouse or human origin. In a specific, non-limiting example, the Fc may be the mouse Fc2b fragment or human Fc1 fragment (Bell et al., 2010; Iqbal et al., 2010). In some embodiments, the Fc domain is from a human immunoglobulin. In additional embodiments, the Fc domain is from a human IgG immunoglobulin. In further embodiments, the Fc domain is from a human IgG1 immunoglobulin. In yet additional embodiments, the Fc domain used can be modified to reduce, for example, its effector function.

The sdAb of the present disclosure can be provided as a chimeric protein. As used in the context of the present disclosure, the terms “chimeric protein” or “chimera” refer to a first proteinaceous entity (e.g., the single domain antibody) which is associated with another (second) entity, which may be proteinaceous as well. The first proteinaceous entity does not naturally occur in association with the second entity. The first proteinaceous entity is modified (via genetic or chemical means) to be capable of associating or be associated with the second entity. The first and second entity may be derived from the same species or the same genera or can be derived from different species or different genera. The first and second entity can be derived from the genera or the species intended to receive the single domain antibody or the chimeric protein. For example, the first and/or the second proteinaceous entity can be derived from humans if the single domain antibody or the chimeric protein are intended to be administered to humans.

In the chimeric proteins of the present disclosure, the single domain antibody can be associated to a carrier. The term “carrier”, as used herein, refers to a molecule that is capable of being associated (covalently or non-covalently, directly or indirectly) with the single domain antibody. The carrier can be physiologically acceptable. In an embodiment, the carrier is immunologically inert, e.g., it lacks the ability to elicit an immune response. The carrier does not substantially interfere with the binding specificity and/or affinity of the single domain antibody to CA-IX. In certain conditions, the carrier can modestly lower the binding affinity of the single domain antibody present in the chimeric protein when compared to the free form single domain antibody (not included in a chimeric protein). In some embodiments, the carrier has a longer clearance time in the blood stream than the single domain antibody alone. It is known in the art that carriers having a molecular weight equal to or higher than 40 kDa (or even higher than 60 kDa) are less rapidly expelled by the kidney and, consequently, have a longer half-life in blood than molecules or smaller size (such as the monovalent antibody moiety described herein).

In the context of the present disclosure, a linker is a chemical entity (which may be proteinacous in nature) covalently associating the single domain antibody with the carrier. The linker may be a releasable or a non-releasable linker. The linker can be a bivalent linker. Alternatively, the linker can comprise multiple bivalent linkers. In one embodiment, the linker includes one or more spacer linkers. In one embodiment, the linker is a peptide linker. The peptide linker can be made of flexible residues (e.g. glycine-serine linkers) such that the linker allows movement of the single domain antibody relative to the carrier. In the context of the present disclosure, the linker is non-immunogenic. In an embodiment, the linker is composed of one or more amino acid residues located between the single domain antibody and the carrier moiety of the chimeric protein. The linker may be of any suitable length to allow for the operable linking of the single domain antibody to the carrier moiety chimeric protein. This embodiment is especially useful when the chimeric protein is intended to be produced in a living organism using a genetic recombinant technique.

The term “biological activity” as used herein in the context of CA-IX includes the biological functions of CA-IX such as the catalysis of the physiological reaction: carbon dioxide hydration to bicarbonate and a proton: CO₂+H₂O↔+H₂CO₃↔H⁺+HCO₃⁻. This reaction is mediated by the catalytic domain of the protein. Moreover, indirectly, this reaction contributes to local acidification. Specifically, it alters the extracellular matrix degradation and the focal adhesion reinforcements which altogether contribute to cell migration and invasion. Thus the biological function of CA-IX includes modulating the acid/base balance inside and outside of the cell expressing CA-IX and therefore cell viability and proliferation.

“Adjuvant therapy” as used herein refers to therapy given in the context of definitive surgery (generally after surgery), where no evidence of residual disease can be detected, so as to reduce the risk of disease recurrence. The goal of adjuvant therapy is to limit or prevent recurrence of the cancer, and therefore to reduce the chance of cancer-related death. Adjuvant therapy can also be administered prior to definitive surgery (e.g. to weaken the tumour and facilitate surgery). “Definitive surgery” as used herein refers to the complete removal of a solid tumor and optionally the surrounding tissue as well as any involved lymph nodes. Examples of such surgery include but are not limited to lumpectomy, mastectomy, such as total mastectomy plus axillary dissection, double mastectomy, colectomy, and nephrectomy.

### DETAILED DESCRIPTION

CA-IX is a major effector of the HIF-1-mediated transcriptional response to tumor hypoxia with a critical role in tumor progression. It is generally exclusively expressed in the hypoxic regions of many types of solid tumors, while mostly absent in normal tissues. Due to its tumor specific expression pattern CA-IX is a marker of poor prognosis across a wide spectrum of solid cancers. Without wishing to be bound by theory, CA-IX is a critical, hypoxia-induced functional effector of several biological processes necessary for cancer growth and metastasis, including pH regulation and cell survival, migration and invasion, maintenance of cancer stem cell (CSC) function, development of the pre-metastatic niche and acquisition of chemo and radioresistance. Importantly, CA-IX's contribution to these processes are primarily controlled by its catalytic activity and the production of H⁺, which, in part, drives acidosis within the extracellular environment and facilitates local invasion through disruption of the extracellular matrix and activation of metalloproteases. The catalytic domain of CA-IX plays a role in maintaining these properties. Furthermore, CA-IX extracellular membrane-bound located catalytic domain makes CA-IX a good target for cancer imaging, therapy and/or diagnosis. The development of therapeutics that selectively inhibit tumor associated, extracellular CAs without “off-target” inhibition of intracellular CAs such as CAII is critical for their use as cancer therapeutics. CA-IX is expressed (and in some embodiments overexpressed) in many solid cancer types. The cancer in which CA-IX can be expressed or overexpressed can be, for example, a lung cancer (such as, for example, a non-small-cell lung cancer or a small-cell cancer), a breast cancer, a liver cancer (such as, for example, an hepatocellular carcinoma), a kidney/renal cancer (such as, for example, a renal cell carcinoma), a stomach cancer, a colorectal cancer, a head and neck tumor, an ovarian cancer, a bladder cancer, a skin cancer (such as, for example, a squamous cell carcinoma, a basal cell carcinoma, a Merkel cell carcinoma, a cutaneous melanoma or a uveal melanoma), an esophagus cancer, a fallopian tube cancer, a genitourinary tract cancer (such as, for example, a transitional cell carcinoma or an endometrioid carcinoma), a prostate cancer (such as, for example, a hormone refractory prostate cancer), a stomach cancer, a nasopharyngeal cancer (such as, for example, a nasopharyngeal carcinoma), a peritoneal cancer, an adrenal gland cancer, an anal cancer, a thyroid cancer (such as, for example, an anaplastic thyroid cancer), a biliary cancer (such as, for example, cholangiocarcinoma), a gastro-intestinal cancer, a mouth cancer, a nervous system cancer, a penis tumor and/or a thymic cancer. In one embodiment, the cancer is not a hematological malignancy as CA-IX may not be expressed or overexpressed in these malignancies. The cancer can be a stage I cancer, a stage II cancer, a stage III cancer or a stage IV cancer. The cancer can be a metastatic cancer. The cancer can be a hormone-sensitive or a hormone-refractory cancer. In one embodiment, the solid tumour cancers that present at least one hypoxic region and are CA-IX positive include but are not limited to breast cancer, colorectal cancer, and renal cancer (such as renal cell carcinoma or clear cell renal cell carcinoma).

For selective and specific binding to the active site in the catalytic domain of hCA-IX, and optionally for inhibition of the catalytic site, single domain antibodies (sdAb) are described herein. The sdAb of the present disclosure can be used as therapeutics as well as diagnostic agents. Single domain antibodies provide certain advantages over the use of monoclonal antibodies, which, as shown herein, can improve the inhibitory function upon binding the catalytic domain of CA-IX. Without wishing to be bound to theory, their small size (e.g., about 15 kDa) and their modularity facilitates a better access to the catalytic pocket of the enzyme while at the same time, unlike SMIs, providing high epitope binding specificity, thus reducing off-target toxicity. There is therefore provided a single domain antibody that specifically targets the catalytic domain of CA-IX. The single domain antibody optionally displays CA-IX enzyme inhibiting qualities but also, optionally, the potential for internalization which, in addition, would allow for bringing cytotoxic payloads into tumor cells.

The single domain antibodies according to the present disclosure are specific and have high affinity to the catalytic domain of CA-IX. The expression “specific to the catalytic domain” as used herein, means the affinity of the sdAbs for the CA-IX catalytic domain is higher than for other polypeptides (for example other CA enzymes or other domains of CA-IX). In some embodiments, the expression “having a high affinity for the catalytic domain” as used herein, refer to the fact that the sdAbs of the present disclosure have a dissociation constant (K_{D}) of 1×10⁻⁷ or lower for a monomeric form of CA-IX and/or a dimeric form of CA-IX. In an embodiment, the antibodies of the present disclosure can recognize and bind to human CA-IX (as described in Gene ID: 768), the mouse CA-IX (as described in Gene ID: 230099), the rat CA-IX (as described in Gene ID: 313495), the monkey CA-IX (Gene ID: 768), and/or the dog CA-IX (Gene ID: 611933). In an embodiment, the antibodies of the present disclosure can recognize and bind to human CA-IX (as described in Gene ID: 768), the mouse CA-IX (as described in Gene ID: 230099), the monkey CA-IX (Gene ID: 768), and/or the dog CA-IX (Gene ID: 611933). In an embodiment, the antibodies of the present disclosure exclusively recognize and bind to the catalytic domain of the human CA-IX (as described in Gene ID: 768).

Therefore, in one embodiment the sdAbs of the present disclosure binds with high specificity and affinity to the catalytic domain of CA-IX. In a further embodiment, the sdAbs, by binding the catalytic domain of CA-IX, inhibit the function of that catalytic domain. In yet a further embodiment, the inhibition of the catalytic function of CA-IX results in a therapeutic effect with respect to CA-IX expressing cancers. Finally, in an additional embodiment, the sdAb is internalized by the cell expressing the CA-IX it binds to, which may be particularly useful for the delivery of a toxic agent to cancerous cells.

In one embodiment, the single domain antibody has a dissociation constant (K_{D}) for a monomeric form of CA-IX and/or a dimeric form of CA-IX of about 1×10⁻⁷ or lower, of about 9×10⁻⁸ or lower, of about 8×10⁻⁸ or lower, of about 7×10⁻⁸ or lower, of about 6×10⁻⁸ or lower, of about 5×10⁻⁸ or lower, of about 4×10⁻⁸ or lower, of about 3×10⁻⁸ or lower, of about 2×10⁻⁸ or lower, or of about 1×10⁻⁸ or lower. In one embodiment, the single domain antibody has a K_{D} for a CA-IX fragment lacking a proteoglycan-like (PG) domain of about 9×10⁻⁹ or lower, of about 1×10⁻¹⁰ or lower, of about 2×10⁻¹⁰ or lower, of about 3×10⁻¹⁰ or lower, of about 4×10⁻¹⁰ or lower, of about 5×10⁻¹⁰ or lower, of about 6×10⁻¹⁰ or lower, of about 7×10⁻¹⁰ or lower, or of about 8×10⁻¹⁰ or lower. As known in the art, CA-II, CA-IV, CA-XII, and CA-IV do not contain a PG domain.

In some embodiments, the single domain antibody is capable of reducing and in some embodiments inhibiting the biological activity of CA-IX. The biological activity of CA-IX is most crucial under hypoxic conditions, for example at the core of a tumor, to maintain the viability of the cells. The activity of CA-IX may be measured, for example, by standard enzyme inhibition assays using purified CA-IX and Methylumbelliferyl acetate (4Mu-Ac) as substrate while measuring fluorescence upon its cleavage in solution by CA-IX, or a in electrometric pH assay as described in Wilbur et al., 1948. The activity may also be measured indirectly, for example by assessing the cell viability, cell proliferation rate or cell death rates of a CA-IX positive cell population in hypoxic conditions. Further examples of indirect indications that CA-IX activity is limited or inhibited are a decrease in tumor growth, a decrease in metastasis potential, and a decrease in cell migration and invasion. For example, the biological activity may be completely eliminated, or eliminated by at least about 50%, at least about 60%, at least about 70%, at least about 80%, or at least about 90%. Furthermore, the single domain antibody can optionally be internalized by a cell expressing CA-IX such as, for example, a cancer cell. In some embodiments, the sdAbs of the present disclosure are as effective to inhibit the biological activity of CA-IX (either in vitro or in vivo) as known small molecule inhibitors of CA-IX, such as, for example SMI U104.

In some embodiments where the single domain antibodies of the present disclosure provide inhibition of the activity of CA-IX, the single domain antibodies of the present disclosure can be used to impede or limit the biological activity of CA-IX in a cell (in vitro or in vivo). In some embodiments, the inhibition of CA-IX can also impede or limit a function in a cell (in vitro or in vivo). In some specific embodiments, the sdAbs of the present disclosure is used to impede the growth or dissemination of a cancer cell. In one example, the single domain antibody is CA9-2-V_{H}H which can impede or limit the CA-IX activity as well as being internalized by the cells. In a further example, the single domain antibody which can impede or limit CA-IX activity as well as being internalized by CA-IX expressing cells consists or comprises SEQ ID NO: 5. In yet further example the single domain antibody which can impede or limit CA-IX activity as well as being internalized by CA-IX expressing cells comprises three CDRs of SEQ ID NO: 6, 7, and 8.

In some embodiments, the sdAbs of the present disclosure can be internalized by a cell (such as a cancer cell) expressing or overexpressing CA-IX. In such embodiments, the sdAbs of the present disclosure can be labelled to detect cells expressing or overexpressing CA-IX. In other embodiments, the sdAbs of the present disclosure can be coupled to a toxic payload to deliver same to a cell expressing or overexpressing CA-IX. In such embodiments, the single domain antibodies of the present disclosure can be associated (coupled or physically linked) to a toxic load and used in combination with a method for the treatment of CA-IX positive cancer cells, such as adjuvant therapy. For example, a toxic load can be biotinylated such that it can be conjugated to the single domain antibodies. In one embodiment, the toxic load can be a chemotherapeutic agent, a toxin (e.g., an enzymatically active toxin of bacterial, fungal, plant, or animal origin, or fragments thereof), a radioactive isotope (i.e., a radioconjugate). Exemplary toxins include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin mertansine (DM1), emtansine, the calicheamicins, and the tricothecenes.

In some embodiments, the single domain antibodies of the present disclosure have one or more complementary determining region comprising or consisting essentially of the amino acid sequence of SEQ ID NO: 2, 3, 4, 6, 7, 8, 10, 11, or 12 (including variants and fragments). In some additional embodiments, the single domain antibodies of the present disclosure have a first complementary determining region comprising or consisting essentially of the amino acid sequence of SEQ ID NO: 2, 6 and 10 (including variants and fragments, a second complementary determining region comprising or consisting essentially of the amino acid sequence of SEQ ID NO: 3, 7 or 11 (including variants and fragments) and/or a third complementary determining region comprising or consisting essentially of the amino acid sequence of SEQ ID NO: 4, 8, or 12. In some further embodiments, the single domain antibodies of the present disclosure have the complementary determining region comprising or consisting essentially of the amino sequence of SEQ ID NO: 2, 3, 4, 6, 7, 8, 10, 11, or 12 (including variants and fragments) as well as any combinations thereof. Moreover, in one embodiment the single domain antibodies of the present disclosure have the first complementary determining region comprising or consisting essentially of the amino acid sequence of SEQ ID NO: 2, a second complementary determining region comprising or consisting essentially of the amino acid sequence of SEQ ID NO: 3, and a third complementary determining region comprising or consisting essentially of the amino acid sequence of SEQ ID NO: 4. Further, in one embodiment the single domain antibodies of the present disclosure have the first complementary determining region comprising or consisting essentially of the amino acid sequence of SEQ ID NO: 6, a second complementary determining region comprising or consisting essentially of the amino acid sequence of SEQ ID NO: 7, and a third complementary determining region comprising or consisting essentially of the amino acid sequence of SEQ ID NO: 8. Furthermore, in one embodiment the single domain antibodies of the present disclosure have the first complementary determining region comprising or consisting essentially of the amino acid sequence of SEQ ID NO: 9, a second complementary determining region comprising or consisting essentially of the amino acid sequence of SEQ ID NO: 10, and a third complementary determining region comprising or consisting essentially of the amino acid sequence of SEQ ID NO: 11.

In the context of the present disclosure, and especially when referred to an amino acid sequence of a complementary determining region, the expression “consisting essentially of” indicates that the sequence in question necessarily comprises the amino acid sequence recited, but that additional, non-essential, amino acid residues can be added at the amino or the carboxyl end of those sequences (as long as these amino acid residues do not substantially modify the intended biological activity of the CDR).

The single domain antibody of the present disclosure can include a variant of a CDR having the amino acid sequence of SEQ ID NO: 2, 3, 4, 6, 7, 8, 10, 11, or 12. In some embodiments, the overall charge, structure or hydrophobic-hydrophilic properties of the single domain antibody can be altered without adversely affecting a biological activity. Accordingly, the amino acid sequence of the CDR can be altered, for example to render the antibody more hydrophobic or hydrophilic, without adversely affecting the biological activities of the single domain antibody. In one embodiment, the variant is a substantially identical sequence to the sequence it is based on.

The single domain antibody of the present disclosure can include a fragment of a CDR having the amino acid sequence of SEQ ID NO: 2, 3, 4, 6, 7, 8, 10, 11, or 12. The CDR fragments can comprise some consecutive amino acid residues of the CDR of amino acid sequence of SEQ ID NO: 2, 3, 4, 6, 7, 8, 10, 11, or 12. In some embodiments, the CDR fragments corresponding to the truncation of one or more amino acid residue at the N- and/or the C-terminus of the wild-type CDR.

In one example, the single domain antibody of the present disclosure has at least three CDRs, where the first CDR comprises or consists of an amino acid sequence of SEQ ID NO: 2, 6, or 10 or a variant or fragment thereof, the second CDR comprises or consists of an amino acid sequence of SEQ ID NO: 3, 7, or 11 or a variant or fragment thereof, and the third CDR comprises or consists of an amino acid sequence of SEQ ID NO: 4, 8, or 12 or a variant or fragment thereof. The sdAbs of the present disclosure can be presented in their monomeric form. In some embodiments, the sdAbs described herein as well as their respective variants or fragments thereof of the present disclosure may also comprise additional sequences to aid in expression, detection or purification of a recombinant antibody or fragment thereof. Any such sequences or tags known to those of skill in the art may be used. For example, and without wishing to be limiting, the antibody or fragment thereof may comprise a targeting or signal sequence (for example, but not limited to ompA), a detection/purification tag (for example, but not limited to c-Myc, His₅, or His₆), or a combination thereof. In another example, the additional sequence may be a biotin recognition site such as that described in WO 95/04069 or in WO/2004/076670. As is also known to those of skill in the art, linker sequences may be used in conjunction with the additional sequences or tags, or may serve as a detection/purification tag.

The single domain antibody, variant or fragment thereof of the present disclosure may also be in a multivalent display format, also referred to herein as multivalent presentation or a multimer. The single domain antibody, variant or fragment thereof of the present disclosure may also be in the form of a chimeric protein.

The present disclosure also provides nucleotide molecules encoding the single domain antibodies, the nanobodies and/or the CDRs described herein. The nucleotide molecules can be provided in an isolated form and may be derived from a variety of sources including DNA, cDNA, synthetic DNA, synthetic RNA, derivatives, mimetics or combinations thereof. Such sequences may comprise genomic DNA, which may or may not include naturally occurring introns, genic regions, non-genic regions, and regulatory regions. Moreover, such genomic DNA may be obtained in association with promoter regions or poly (A) sequences. The sequences, genomic DNA, or complementary DNA (cDNA) may be obtained in any of several ways. Genomic DNA can be extracted and purified from suitable cells by means well known in the art. Alternatively, mRNA can be isolated from a cell and used to produce cDNA by reverse transcription or other means. The nucleotide molecules described herein are used in certain embodiments of the methods of the present disclosure for production of RNA, proteins or polypeptides, through incorporation into host cells, tissues, or organisms. In an embodiment, the nucleotide molecules can be codon-optimized for expression in a particular host. The nucleotide molecules can include, in some embodiments, one or more promoter sequence and/or one or more terminator sequence. The nucleotide molecules can be included in a vector for expression in a recombinant host. The nucleotide molecules of the present disclosure can include, in some embodiments, the nucleic acid sequence of SEQ ID NO: 21, 22 or 23, variants thereof or fragments thereof.

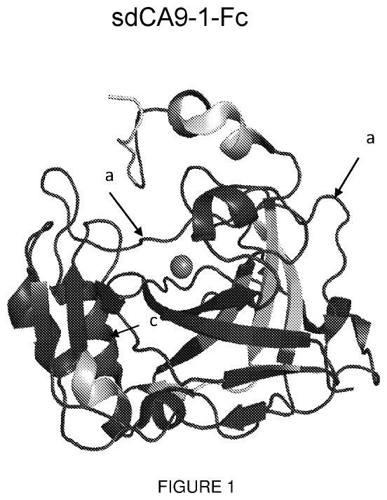

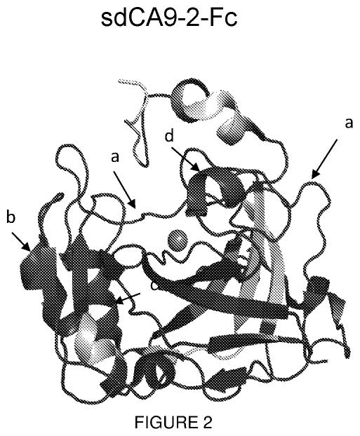

The single domain antibody according to the present disclosure can bind the epitope (in the catalytic domain of CA-IX) as a conformational epitope or a linear epitope. FIGS. 1 and 2 illustrate two examples of binding conformations of the sdAb with CA-IX. FIG. 1 shows the binding of the specific example CA9-1-Fc single domain antibody and FIG. 2 the binding of the specific example CA9-2-Fc single domain antibody. The examples of the present disclosure expand on the properties of CA9-1-Fc and CA9-2-Fc. FIG. 1 shows the CA-IX in its bound confirmation 1 (i.e. with a stabilized epitope) with zinc 4 at the core. The epitope in FIG. 1 is a linear epitope 3 that is essentially situated at the alpha helix identified in FIG. 1. The epitope for CA9-1-Fc is therefore a linear epitope that is limited to one side of the catalytic groove of CA-IX. On the other hand, FIG. 2 shows the CA-IX in its bound conformation 2 (i.e. with a stabilized epitope) that spans to a larger portion of the catalytic groove. Indeed, the conformational epitope 5 includes the same alpha helix as in FIG. 1, as well as the two neighbouring beta sheets and loop, as well as the loop situated above the zinc 4. The epitope for CA9-2-Fc is therefore a conformation epitope that spans across both sides of the catalytic grooves. Without wishing to be bound by theory, it is thought that conformation epitopes are more likely to provide inhibition to the catalytic activity of CA-IX because the Zn interaction is disrupted which destabilizes the three dimensional conformational structure of the catalytic domain. It is contemplated within the scope of the present disclosure, that the single domain antibodies have an epitope located at the catalytic domain (linear or conformation). The scope thus includes many possible epitopes other than the two exemplary epitopes described herein to illustrate the differences between a linear and conformation epitope in the context of the catalytic domain of CA-IX. In one embodiment, the sdAb is one or more of CA9-1-V_{H}H, CA9-2-V_{H}H, and CA9-3-V_{H}H as described in the Example.

The single domain antibodies of the present disclosure can be used to detect, and in some embodiments, localize or quantify the amounts of CA-IX either in vitro (in immunological assays, such as, for example, ELISA, immunological staining and flow cytometry) or in vivo (in imaging techniques). In one embodiment, the single domain antibodies accordingly to the present disclosure are provided to the target site. The target site may be in an in vitro setting or an in vivo setting. In the in vitro case, the environment can be controlled and the single domain antibodies can be reliably provided directly to the target cells. In the in vivo case, the single domain antibodies may be directly injected to reach the target cells if they are readily accessible or can be indirectly provided through the blood stream (such as intravenous injection). Once the single domain antibodies are in the target site, diffusion will necessarily bring the at least a portion of the single domain antibodies to CA-IX (if it is expressed by the target cells). If CA-IX is present at the target site, the single domain antibodies will therefore bind to the catalytic domain of CA-IX and form a complex with CA-IX. In such embodiments, the single domain antibodies of the present disclosure can be associated (coupled or physically linked) to a detectable label and used in combination with a method for detecting, localizing and/or quantifying the amount of CA-IX enzyme by determining the presence, absence, location, amount of the detectable label. The single domain antibodies in imaging methods can be provided in an imaging effective amount. The term “imaging effective amount” as used herein refers to an amount of an imaging agent that allows the detection of the target species (such as CA-IX) above a certain threshold of target species concentration. To facilitate the detection of the complex, the single domain antibodies can be coupled with a detectable label. Examples of detectable labels include, but are not limited to, various enzymes, prosthetic groups, fluorescent materials, luminescent materials, bioluminescent materials and radioactive materials. Examples of suitable enzymes include, but are not limited to, horseradish peroxidase, alkaline phosphatase, beta-galactosidase, or acetylcholinesterase. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride or phycoerythrin. Examples of a luminescent material include, but are not limited to, luminol. Examples of bioluminescent materials include, but are not limited to, luciferase, luciferin, and aequorin. Examples of suitable radioactive materials include, but are not limited to, ¹²⁵I, ¹³¹I, ³⁵S, ³²P or ³H. Once the complex is formed between the single domain antibodies of the present disclosure and CA-IX, a detection method adapted to the detectable label can be used to identify and quantify the complexes. A control can be used to determine accurately the presence or absence or location of CA-IX expressing cells. For example, the control can be one or more of a single domain antibody that is not specific to CA-IX or a single domain antibody lacking the detectable label. In preferred embodiments, the single domain antibodies selected for imaging can be internalized by CA-IX expressing cells. The internalization can improve in vivo as well as in vitro imaging. Indeed, as the detectable label is associated with the single domain antibodies, the detectable label can be internalized by the cells. This specific embodiment, may advantageously allow to image the hypoxic sections of a solid tumor. Depending on the detectable label and the detection method, the image capture can be performed shortly after injection of the single domain antibodies or may require a wait time. In some embodiments, prolonged imaging may be advantageous. For example, the tumor can be mapped and imaged to identify the specific cancerous cells and thus guide the excision of the solid tumor thereby potentially reducing the quantity of healthy tissue cut along with the solid tumor. The present imaging methods can be performed to a subject having received a cancer diagnosis or to a subject where cancer has not yet been identified. The imaging methods according to the present disclosure can contribute to the detection, confirmation, and/or identification of cancer.

The single domain antibody of the present disclosure can be provided in a chimeric form. In one embodiment, the chimeric form may provide further applications of the single domain antibodies. For example, the single domain antibody can be provided in a chimeric form in immunotherapeutic molecules, such a chimeric antigen receptor (CAR) construct or a bispecific T cell engager (BiTE) construct. The CAR and/or BiTE constructs can be used, in some embodiments, for T-cell therapy and in yet further embodiments, for the treatment of cancer (such as, for example, for the treatment of a hematological cancer. In yet another example, the single domain antibody can be provided in a multimeric form by presenting a plurality of single domain antibodies (which may be the same or different) on a carrier.

In some embodiments, the two entities of the chimeric protein can be associated together prior to the administration to a recipient. The two entities can also be associated only after the single domain antibody moiety is administered to the recipient. The association between the two moieties can be covalent or non-covalent and can occur prior to, during or after administration.

The small size of the single domain antibodies is an advantage and improves the access of the CDRs to the epitope on the catalytic pocket of CA-IX. However, the small size can be disadvantage when prolonged bioavailability is desired. Thus, in an embodiment, the carrier can be an entity to improve the circulation time and bioavailability. In an embodiment, the carrier is a protein, including, but not limited to a plasma protein. Plasma proteins include, but are not limited to serum albumin, immunoglobulins fragments (provided that these fragments do not or substantially do not affect the binding affinity of the single domain antibody), alpha-1-acid glycoprotein, transferrin, or lipoproteins. When an immunoglobulin fragment is used, it can be obtained from any immunoglobulin. In a specific embodiment, the immunoglobulin fragment is derived from a human immunoglobulin (such as, for example, IgA, IgD, IgG, IgE or IgM). In yet a further embodiment, the immunoglobulin fragment is derived from a IgG antibody (such as, for example, IgG1, IgG2, IgG3 or IgG4). In a specific embodiment, the carrier can be a Fc which may have been modified to no longer have an effector function. In a further specific embodiment, the carrier can comprise or consist of the amino acid sequence of SEQ ID NO: 15, a variant thereof or a fragment thereof. In some instances, it is contemplated that a human protein, such as a human plasma protein be used as the carrier. This embodiment is particularly useful when designing therapeutics for the treatment of humans or for making a chimeric protein in which the monovalent antibody moiety is derived (directly or indirectly) from a human antibody or a humanized antibody. In an embodiment, the carrier is an immunoglobulin fragment, such as a single domain antibody moiety. In another embodiment, the carrier is not proteinaceous in nature, but is rather a chemical polymer. Such polymers include, but are not limited to, PEG.

In some embodiments, the chimeric protein can be designed to allow, facilitate or increase the translocation of the single domain antibody of the present disclosure.

In the chimeric protein, the single domain antibody can be associated directly to the carrier. Alternatively, the monovalent antibody moiety can be associated indirectly to the carrier by using one or more linkers between the single domain antibody and the carrier The amino acid linker can comprise one or more amino acid residues. For example, the amino acid linker can comprises one or more glycine residues such as an hexa-glycine linker. In another example, the amino acid linker can comprise or consist of the amino acid sequence of SEQ ID NO: 14, a variant thereof or a fragment thereof. The present chimeric protein also includes those using a non-amino acid linker, such as a chemical linker. The chimeric polypeptide may be a single-chain polypeptide comprising any suitable peptide linker, wherein a suitable peptide linker may comprise any amino acid sequence that allows for the linked components of the fusion protein to maintain their unrestricted desired biological function. For example, the peptide linker may comprise a sequence of glycine or serine residues or may be any suitable peptide linking sequence. In some instances, the chimeric protein is exclusively made of amino acids and is produced by a living organism using a genetic recombination technique. The chimeric protein can consist of a single domain antibody, a carrier and an amino acid linker.

The single domain antibody can be associated with the linker or the carrier moiety of the chimeric protein at any amino acid residue(s), provided that the association does not impede the single domain antibody from binding to the catalytic domain of CA-IX. In some instances, the linker or the carrier is associated to one or more amino acid residue(s) of the single domain antibody that is (are) not involved in specifically binding the catalytic domain of CA-IX. In some instances, the linker or the carrier is associated to a single amino acid residue of the single domain antibody. The linker or the carrier can be associated with any amino acid residue of the single domain antibody, including the amino acid residue located at the amino-terminus of the single domain antibody or at the carboxyl-terminus of the single domain antibody. In instances in which the linker and the carrier are also of proteinaceous nature, the single domain antibody can be associated to any amino acid residue of the linker or the carrier, including the amino acid residue located at the amino-terminus of the linker or the carrier or the amino acid residue located at the carboxyl-terminus of the linker or the carrier. In an embodiment, the amino acid residue located at the amino-terminus of the linker or the carrier is associated to the amino acid residue located at the carboxyl-terminus of the single domain antibody. In still another embodiment, when the linker is present and is of proteinaceous nature, its amino terminus is associated to the carboxyl terminus of monovalent antibody and its carboxyl terminus is associated with the amino terminus of the carrier.

In instances where a covalent association is sought between the single domain antibody and the carrier, the association between the two entities can be a peptide bond. Such embodiment is especially useful for chimeric proteins wherein the at least two entities are both proteinaceaous and are intended to be produced as a fusion protein in an organism (prokaryotic or eukaryotic) using a genetic recombinant technique. Alternatively, the covalent association between the two moieties can be mediated by any other type of chemical covalent bounding. In some instances, the chimeric proteins are designed so as not to be susceptible of being cleaved into the two moieties in the general circulation (for example in plasma).

As indicated above, the association between the two entities can be non-covalent. Exemplary non-covalent associations include, but are not limited to the biotin-streptavidin/avidin system. In such system, a label (biotin) is covalently associated to one entity/moiety while a protein (streptavidin or biotin) is covalently associated with the other entity/moiety. In such embodiment, the biotin can be associated to the single domain antibody or to the carrier, providing that the other entity in the system is associated with streptavidin or avidin.

In a further system of non-covalent association, the first entity is designed to be non-covalently associated to the second entity only upon its administration into the intended recipient. This embodiment is especially useful when the carrier is a protein present in the blood of the recipient. For example, single domain antibody may be associated (in a covalent or a non-covalent fashion) with a second antibody, a lectin or a fragment thereof (referred to herein as an antibody-derived linker) which is capable of non-covalently binding the carrier once administrated to the intended recipient. For example, the second antibody, lectin or fragment thereof can be specific for any blood/plasma protein present in the intended recipient (such as, for example, serum albumin, immunoglobulins fragments (provided that these fragments do not affect or do not substantially affect the binding of the single domain antibody to the catalytic domain of CA-IX), alpha-1-acid glycoprotein, transferrin, or lipoproteins). The second antibody, lectin or fragment thereof can be associated, preferably in a covalent manner, with the single domain antibody at any amino acid residue of the single domain antibody, but preferably at the amino- or carboxyl-end of the monovalent antibody moiety. In such embodiment, the second antibody, lectin or fragment thereof is akin to a linker between the single domain antibody and the carrier. Upon the administration of this embodiment of the single domain antibody in a recipient, the carrier (a blood or plasma protein for example) associates with the second antibody, lectin or fragment thereof to form, in vivo, the chimeric protein. In a specific embodiment, the second antibody is an antibody specifically recognizing albumin (such as, for example, an antibody specifically recognizing human albumin).

In a specific embodiment, the chimeric polypeptide can comprise or consist of the amino acid sequence of SEQ ID NO: 16 to which the leader sequence may have been removed. In such embodiment, the chimeric polypeptide can be encoded by a nucleic acid molecule comprising or consisting of the nucleic acid sequence of SEQ ID NO: 24 to which the sequence encoding the leader sequence may have been removed. In a specific embodiment, the chimeric polypeptide can comprise or consist of the amino acid sequence of SEQ ID NO: 17 to which the leader sequence may have been removed. In such embodiment, the chimeric polypeptide can be encoded by a nucleic acid molecule comprising or consisting of the nucleic acid sequence of SEQ ID NO: 25 to which the sequence encoding the leader sequence may have been removed. In a specific embodiment, the chimeric polypeptide can comprise or consist of the amino acid sequence of SEQ ID NO: 18 to which the leader sequence may have been removed. In such embodiment, the chimeric polypeptide can be encoded by a nucleic acid molecule comprising or consisting of the nucleic acid sequence of SEQ ID NO: 25 to which the sequence encoding the leader sequence may have been removed.

The single domain antibody or the chimeric protein comprising same can be formulated for administration with an excipient. An excipient or “pharmaceutical excipient” is a pharmaceutically acceptable solvent, suspending agent or any other pharmacologically inert vehicle for delivering one or more chimeric protein to a subject, and is typically liquid. A pharmaceutical excipient is generally selected to provide for the desired bulk, consistency, etc., when combined with components of a given pharmaceutical composition, in view of the intended administration mode. Typical pharmaceutical excipients include, but are not limited to binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycotate, etc.); and wetting agents (e.g., sodium lauryl sulphate, etc.).

In one embodiment, the single domain antibodies of the present disclosure can be combined to form a multivalent chimera. The term “multivalent” as used herein, refers to a system having two or more single domain antibodies each having variable regions that recognize a different epitope. In one embodiment, the two epitopes are different. In a further embodiment, the two epitopes are on different molecules thereby rendering the multivalent chimera potentially multifunctional. In yet a further embodiment, the two epitopes are different but on the same molecule. As described above for chimeras, a linker can be used to create a multivalent antibody comprising the single domain antibody of the present disclosure. The three dimensional conformation of a multivalent chimera can be optimized for optimal binding of the epitopes. For example, depending on the epitope conformations a multivalent chimera can be linear i.e. the first single domain antibody, the linker, and the second single domain antibody are on the same line, and thus present an angle of about 1800 between them. In other embodiment, the linker can positioned such that the single domain antibodies are perpendicular to each other. Many different conformations are contemplated for the present single domain antibody, especially for multifunctional chimera. The angle between can be varied as needed to be optimized for a specific application and epitopes. For example, the linker can bind the two moieties such that they are positioned one on top of the other, and the angle between them can be varied from acute to obtuse. In one embodiment, the CDRs according to the present disclosure can be grafted onto a multivalent antibody. Accordingly, in one embodiment, a multivalent antibody has one or more complementary determining region comprising or consisting essentially of the amino acid sequence of SEQ ID NO: 2, 3, 4, 6, 7, 8, 10, 11, or 12 (including variants and fragments). In some additional embodiments, the multivalent antibodies of the present disclosure have a first complementary determining region comprising or consisting essentially of the amino acid sequence of SEQ ID NO: 2, 6 and 10 (including variants and fragments, a second complementary determining region comprising or consisting essentially of the amino acid sequence of SEQ ID NO: 3, 7 or 11 (including variants and fragments) and/or a third complementary determining region comprising or consisting essentially of the amino acid sequence of SEQ ID NO: 4, 8, or 12. In some further embodiments, the multivalent antibodies of the present disclosure have the complementary determining region comprising or consisting essentially of the amino sequence of SEQ ID NO: 2, 3, 4, 6, 7, 8, 10, 11, or 12 (including variants and fragments) as well as any combinations thereof. Moreover, in one embodiment the single domain antibodies of the present disclosure have the first complementary determining region comprising or consisting essentially of the amino acid sequence of SEQ ID NO: 2, a second complementary determining region comprising or consisting essentially of the amino acid sequence of SEQ ID NO: 3, and a third complementary determining region comprising or consisting essentially of the amino acid sequence of SEQ ID NO: 3. Further, in one embodiment the single domain antibodies of the present disclosure have the first complementary determining region comprising or consisting essentially of the amino acid sequence of SEQ ID NO: 6, a second complementary determining region comprising or consisting essentially of the amino acid sequence of SEQ ID NO: 7, and a third complementary determining region comprising or consisting essentially of the amino acid sequence of SEQ ID NO: 8. Furthermore, in one embodiment the single domain antibodies of the present disclosure have the first complementary determining region comprising or consisting essentially of the amino acid sequence of SEQ ID NO: 9, a second complementary determining region comprising or consisting essentially of the amino acid sequence of SEQ ID NO: 10, and a third complementary determining region comprising or consisting essentially of the amino acid sequence of SEQ ID NO: 11.

In other embodiments, the small size of the single domain antibodies is beneficial in the sense that it allows for combining two or three V_{H}Hs to generate multi-functional therapeutic antibodies: multivalent antibodies. These approaches increase the overall size of the therapeutic antibody, and thus leads to improved serum half-life.

In one embodiment, the single domain antibody is generated from a camelid (e.g. camel, llama, etc.) immunization. The binding specificity and functional activity of a single domain antibody produced can be assessed in an enzyme activity assay using purified CA-IX protein and in vitro using cells. The V_{H}Hs obtained from the camelids can also be expressed and produced in bacteria or mammalian cells (CHO), while the Fc-fused V_{H}Hs can be expressed in mammalian cells (CHO). Accordingly, in one embodiment, the single domain antibody is formulated as a vector comprising the nucleic acid sequence of the single domain antibody. In another embodiment, the vector can be transfected into a host cell. The vector can include a promoter and optionally one or more enhancers. Examples of suitable promoters include but are not limited to the lac promoter, the T7 promoter system, a phage promoter (pL promoter). The promoter can be selected to have an inducible expression (chemical, pH, temperature, molecular, etc. induction) or a continuous expression. Therefore, in a further embodiment there is provided a host cell comprising the nucleic acid sequence of the single domain antibodies according to the present disclosure.

The single domain antibody according to the present disclosure can be produced by a method comprising the steps of providing a host cell having a nucleic acid sequence of the single domain antibody, stimulating the production of the single domain antibody, and recovering the single domain antibodies. For example, the nucleic acid sequence can be incorporated into the genome (such as the chromosome) of the host cell. The selection of an appropriate recombinant host cell can vary depending on the recombinant protein produced, to optimize the production rate, efficiency and/or quality. For example the recombinant cell can be a prokaryote (e.g. Escherichia coli) or a eukaryote (e.g. Saccharomyces cerevisiae or Pichia pastoris). Following the production of the recombinant protein, the recombinant protein can be recovered and purified, then formulated into a pharmaceutical composition. Indeed, the single domain antibodies of the present disclosure can be formulated in solution (such as a pharmaceutical composition) or as a solid precursor (such as a powder) to improve the storage and shelf life. The solid precursor can then be added to form or complete a pharmaceutical composition.

A pharmaceutical composition can be produced by generating, providing, or producing a single domain antibody, a multivalent antibody or a chimeric polypeptide according to the present disclosure, and adding a pharmaceutically acceptable excipient. Thus, the single domain antibody, the multivalent antibody or the chimeric polypeptide can be formulated as a pharmaceutical composition for administration with an excipient. An excipient or “pharmaceutical excipient” is a pharmaceutically acceptable solvent, suspending agent or any other pharmacologically inert vehicle for delivering one or more single domain antibody to a subject, and is typically liquid. A pharmaceutical excipient is generally selected to provide for the desired bulk, consistency, etc., when combined with components of a given pharmaceutical composition, in view of the intended administration mode. Typical pharmaceutical excipients include, but are not limited to binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycotate, etc.); and wetting agents (e.g., sodium lauryl sulphate, etc.).

The single domain antibody, the multivalent antibody or the chimeric polypeptide according to the present disclosure may be formulated for administration with a pharmaceutically-acceptable excipient, in unit dosage form or as a pharmaceutical composition. Conventional pharmaceutical practice may be employed to provide suitable formulations or compositions to administer such compositions to subjects. Although intravenous administration is preferred, any appropriate route of administration may be employed, for example, oral, parenteral, subcutaneous, intramuscular, intracranial, intraorbital, ophthalmic, intraventricular, intracapsular, intraspinal, intrathecal, epidural, intracisternal, intraperitoneal, intranasal, or aerosol administration. Therapeutic formulations may be in the form of liquid solutions or suspension. Methods well known in the art for making formulations are found in, for example, Remington: The Science and Practice of Pharmacy, (19th ed.) ed. A. R. Gennaro AR., 1995, Mack Publishing Company, Easton, PA. The administration can be to a subject (human or mammal) or to an in vitro cell culture. The cell culture may be a culture of primary cells cultivated from a human or mammal. The cell culture or the subject preferably have cells that express or overexpress CA-IX and may be in hypoxic conditions. In one embodiment, these cells are cancerous, hypoxic or pre-necrotic.

Because some of the embodiments of the antibodies of the present disclosure are capable of reducing colony formation of cancerous cells, the antibodies (single domain, multivalent, chimeric) can also be used for the treatment or the alleviation of symptoms associated with a hyperproliferative disease. The expressions “treatment or alleviation of symptoms” refer to the ability of a method or an antibody to limit the development, progression and/or symptomology of a hyperproliferative disease. In one embodiment, the treatment and/or alleviation of symptoms can encompass the reduction of proliferation of the cells (e.g., by reducing the total number of cells in a hyperproliferative state and/or by reducing the pace of proliferation of cells). Symptoms associated with proliferation-associated disorders include, but are not limited to: local symptoms which are associated with the site of the primary cancer (such as lumps or swelling (tumor), hemorrhage, ulceration and pain), metastatic symptoms which are associated to the spread of cancer to other locations in the body (such as enlarged lymph nodes, hepatomegaly, splenomegaly, pain, fracture of affected bones, and neurological symptoms), and systemic symptoms (such as weight loss, fatigue, excessive sweating, anemia and paraneoplastic phenomena).

Hyperproliferative diseases form a class of diseases where cells proliferate more rapidly, and usually not in an orderly fashion. The proliferation of cells cause a hyperproliferative state that may lead to biological dysfunctions, such as the formation of tumors (malignant or benign). An example of a hyperproliferative disease is cancer, also known medically as a malignant neoplasm. Cancer is a term for a large group of different diseases, all involving unregulated cell growth. In cancer, cells divide and grow uncontrollably, forming malignant tumors, and invade nearby parts of the body. The cancer may also spread to more distant parts of the body through the lymphatic system or bloodstream. In another embodiment, the cancer is associated with the expression and, in some embodiments overexpression, of the CA-IX (i.e. a CA-IX positive cancer). In some embodiments, the antibodies can be used in combination with other chemotherapeutic agents.

Accordingly, there is provided a method of method of limiting the biological activity of carbonic anhydrase IX (CA-IX) expressed by a cell. The method comprises contacting the single domain antibody, the multivalent antibody, or the chimeric polypeptide according to the present disclosure, with CA-IX expressed by the cell so as to limit the biological activity of CA-IX in the cell when compared to a control cell contacted by a control single domain antibody that fails to specifically bind to the catalytic domain of CA-IX and reduce the biological activity of CA-IX. In one embodiment, the single domain antibody, the multivalent antibody, or the chimeric polypeptide is internalized by the cells through the transmembrane CA-IX. In one example, a Fc fusion to the sdAbs of the present disclosure can improve the internalization. The step of contacting the single domain antibody, the multivalent antibody, or the chimeric polypeptide according to the present disclosure can be performed by an administration of a pharmaceutical composition according to the present disclosure. In one embodiment, the biological activity includes but is not limited to the catalysis of carbon dioxide to bicarbonate, signal transduction, promoting cell survival in hypoxic conditions, promoting or supporting tumorigenesis, promoting drug resistance for conventional cancer therapy drugs (e.g. chemotherapy or radiation therapy), and promoting the metastatic migration of tumor cells. In one embodiment, limiting the biological activity can be defined as reducing the occurrence or rate of one or more of the activities of CA-IX by about at least about 50%, at least about 60%, at least about 70%, at least about 80%, or at least about 90% compared to their occurrence or rate without the administration of a pharmaceutical composition according to the present disclosure. The impact of limiting the biological activity of CA-IX can be observed as the alleviation of a symptom of a cancer, the treatment of a cancer, the prevention of the re-occurrence of cancer, delivery of a toxic payload to the cell, and/or for improving the usefulness of a further therapeutic agent. In one embodiment, the toxic payload is selected from ozogamicin, vedotin, emtansine, anthracycline toxin PNU-159682, maytansinoids (e.g. DM1, DM4), and radio isotopes.

The single domain antibodies with a toxic payload according to the present disclosure can be contacted with CA-IX expressing cells (in vitro or in vivo) or administered to a subject in need thereof in a pharmaceutically effective amount or therapeutically effective amount. These expressions refer to an amount (dose) effective in mediating a therapeutic benefit to a subject (for example reducing immune suppression, increasing immune cytotoxicity, treatment and/or alleviation of symptoms of cancer). It is also to be understood herein that a “pharmaceutically effective amount” may be interpreted as an amount giving a desired therapeutic effect, either taken in one dose or in any dosage or route, taken alone or in combination with other therapeutic agents. The single domain antibodies of the present disclosure can therefore be used to prevent, alleviate the symptoms or treat conditions related to the expression of CA-IX in cells (such as hypoxic cells in tumors).

In one embodiment, there is provided a therapeutic regimen comprising the single domain antibodies, the multivalent antibodies, or the chimeric polypeptide. The therapeutic regimen can further comprise one or more known anti-cancer therapies (such as anti-cancer drugs, immunotherapy, chemotherapy, and the like). Alternatively, the therapeutic regimen can be administered in combination with any known cancer therapeutic regimen. In one embodiment, the therapeutic regimen comprises administering to a subject in need thereof a therapeutically effective amount of the pharmaceutical composition according to the present disclosure. In one embodiment, the single domain antibodies and compositions comprising can be administered as an adjuvant therapy for a cancer subject that has undergone definitive surgery to extend the disease free survival (DFS) or overall survival (OS) in the subject. In one embodiment the pharmaceutical composition can be administered as adjuvant therapy in combination with another therapeutic agent. In one embodiment, the adjuvant therapy according to the present disclosure reduces cancer recurrence. In one embodiment, the cancer cells targeted are in the hypoxic regions of many types of solid tumors and express CA-IX. Examples of solid tumour cancers that present at least one hypoxic region and are CA-IX positive include but are not limited to breast cancer, colorectal cancer, and renal cancer (such as renal cell carcinoma or clear cell renal cell carcinoma).

In one embodiment, the present disclosure provides a method of detecting a cell expressing or overexpressing carbonic anhydrase IX (CA-IX). The method comprises contacting the single domain antibody, the multivalent antibody or the chimeric polypeptide according to the present disclosure with the cell (which is susceptible to express CA-IX) under conditions so as to allow the specific binding of the single domain antibody, the multivalent antibody or the chimeric polypeptide to the cell. The presence of a complex formed between the cell and the single domain antibody or the chimeric polypeptide can then be detected which gives the indication on whether the cell is expressing or overexpressing CA-IX if the presence of the complex is determined to be present. In one embodiment, the cell that is detected is a cancerous cell, an hypoxic cell and/or a pre-necrotic cell. In one embodiment, the cell is present in a subject. In one embodiment, the subject is a human or a mammal. The method may allow for the detection, localization and/or quantification of the amount of CA-IX enzyme by determining the presence, absence, location, and/or amount of the detectable label. Thus the method can involve the steps of determining the enzyme activity of the label, detecting the formation of a prosthetic group complex or imaging or quantifying fluorescent materials, luminescent materials, bioluminescent materials or radioactive materials. Optionally, the results are compared to a standard control sample, or data collected previously from one or more standard controlled samples. The control may be one or more of a single domain antibody that is not specific to CA-IX, a single domain antibody without the detectable label, and a sample that is known to not contain any CA-IX, etc. . . . . Optionally, a control sample is performed concurrently with the imaging analysis and compared with the test sample.

In one embodiment, the single domain antibody, the multivalent antibody or the chimeric polypeptide can be used in imaging solid tumours having cells expressing CA-IX. In one example, nanoparticles can be grafted onto the single domain antibody, the multivalent antibody or the chimeric polypeptide. Other imaging materials such as the detectable labels described herein can be employed in the imaging of solid tumours. Upon stimulation, the nanoparticles emit light of a specific wavelength that is detectable. For example, the light can be detected by fundus photography, angiography, optical coherence tomography (OCT), and/or OCT angiography (OCTA). This wavelength of emitted light provides information on the origin of the specific sessile or particulate circulating in the blood or other fluids to which the plurality of nanoparticles containing specific tumor antibodies are attached. In one embodiment, after stimulation of the quantum dot nanoparticles with various wavelengths of external light, a fluorescein angiography fundus camera records the light emitted by nanoparticles. This provides a non-invasive method of imaging, and also provides a method of early cancer detection and differentiation based on wavelength differentiation, i.e., optical spectroscopy), which may then be treated by a specific therapy. In one embodiment, imaging is combined with nanoparticle assisted photoacoustic imaging, OCT, OCTA, FA, focused ultrasound, non-focused ultrasound, MRI, PET scan, CT scan, surface enhanced Raman spectroscopy and imaging, which enhances the molecular diagnosis of a substance attached to the surface of a metallic nanoparticle after it is exposed to the laser light energy recorded to enhance an early disease diagnosis in vivo.

### Example

Antibody generation. A llama was immunized with the recombinant CA-IX antigen (NRT-HHT-Montreal Production Team; 1.55 mg/ml; mixture of monomer and dimer). Seven injections were performed at approximately two-week intervals and blood was collected at each injection. Total RNA was isolated from lymphocytes collected from day 70 of the immunization and cDNA was transcribed. Based on the Camelidae and llama immunoglobulin databases, three variable domain sense primers (MJ1-3) and two CH2 domain antisense primers (CH2 and CH2b3) were designed (Doyle et al. 2008). The first PCR was performed with the cDNA as template and the variable regions of both conventional (IgG1) and heavy chain antibodies (IgG2 and IgG3) were amplified with combinations of MJ1-3/CH2 and MJ1-3/CH2b primers in two separate reactions. The heavy-chain portion of the amplified PCR products were gel-purified and used in a second PCR reaction using two-VHH specific primers: MJ7 and MJ8. The amplified products (about 400-450 bp) that correspond to VHH fragments of heavy chain antibodies were digested and gel purified.

Library construction. For library construction, digested pMED1 DNA was ligated with the digested V_{H}H fragments. The ligated product was then electroporated into competent E. coli TG1cells (Stratagene, Cedar Creek, TX). The V_{H}H fragments from 30 colonies were PCR-amplified and sequenced for diversity analysis.

Library panning and screening. Panning was performed essentially as described by (Arbabi-Ghahroudi, MacKenzie, and Tanha 2009; Arbabi Ghahroudi et al. 1997). Approximately 1011 of rescued phage from the library were used in each round of panning and after extensive washing, bound phages to the immobilized CA-IX antigen were eluted, amplified in bacteria, and used for the next round of panning (four rounds performed in total). Phage ELISA was performed on the individual colonies (48 in total) obtained after four rounds of panning. Colony-PCR was performed on positive colonies and the PCR products were sent for sequencing. From sequencing data, three unique positive binders were identified sdCA9-1-V_{H}H, sdCA9-2-V_{H}H, and sdCA9-3-V_{H}H-VHH demonstrated strong binding to CA-IX by SPR.

Expression of soluble V_{H}H. Restriction enzyme sites BbsI and BamHI were added to the 5′ and 3′ ends of the positive V_{H}H DNA fragments using a PCR involving gene-specific sense primer V_{H}H BbsI (5′-TATGAAGACACCAGGCCCAGGTGCAGCTGGTGGAGTCT-3′) (SEQ ID NO: 19) and anti-sense primer V_{H}H-BamHI (5′-CGCGGGATCCTGAGGAGACGGTGACCTGGGT-3′) (SEQ ID NO: 20). The amplified DNA was then digested with BbsI and BamHI restriction enzymes and ligated into digested pSJF2 vector using standard cloning techniques. Competent E. coli TG1 cells were transformed and clones harbouring CA-IX-specific recombinant V_{H}H were grown in 1-liter cultures of 2xYT medium+ampicillin (100 mg·mL-1) with 0.1% glucose to an OD600 of 0.8. Cultures were induced with 1 mM IPTG and grown overnight on a rotary shaker at 28° C. After confirmation of expression by SDS-PAGE and Western blotting, recombinant V_{H}H proteins were extracted from the bacterial cells by standard lysis methods and purified by immobilized metal affinity chromatography (IMAC) and quantified. The state of aggregation of the purified protein was checked by size exclusion chromatography on Superdex 200 (Amersham Biosciences). The reactivity of the individual V_{H}H proteins was confirmed by ELISA in which rabbit anti-His₆ antibody conjugated to HRP was used for the detection of binding.

Expression of Fc-fused format. sdCA9 sequences were cloned between the EcoRI and BstEII sites of a pTT5 vector that already contained a V_{H}H-Fc. This results in replacement of the existing V_{H}H with the CA9 V_{H}Hs below as in-frame fusions with human IgG1-Fc. sdCA9-Fc 1 (e.g., having the amino acid sequence of SEQ ID NO: 16, encoded by the nucleic acid sequence of SEQ ID NO: 24), 2 (e.g., having the amino acid sequence of SEQ ID NO: 17, encoded by the nucleic acid sequence of SEQ ID NO: 25) and 3 (e.g., having the amino acid sequence of SEQ ID NO: 18, encoded by the amino acid sequence of SEQ ID NO: 26) were produced in CHO3E7 cells and purified by protA. Purity was evaluated by running on a Commassie gel.

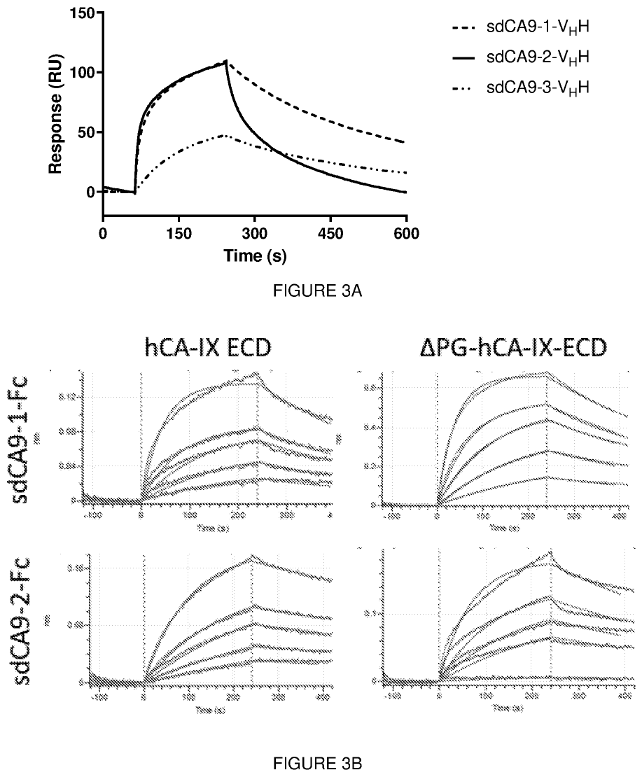

In vitro binding affinity/SPR. To evaluate the binding affinity (K_{D}) of the V_{H}H only format of the 4 single domain antibodies, CA-IX monomer and dimer were immobilized on SA sensor chip (GE Healthcare) and serial dilutions of the sdAbs were injected over the surface (FIG. 3A). The affinity measurement determined by SPR methods suggests that the sdAbs have similar binding affinities to monomeric and dimeric CA-IX that lie within the 1-2 nM range. SPR data revealed that sdCA9-1-V_{H}H and sdCA9-2-V_{H}H bind with high affinity and sdCA9-3-V_{H}H also binds with high affinity but less so than the other two. The two single domain antibodies sdCA9-1-V_{H}H and sdCA9-2-V_{H}H were selected for further characterizations. Table 1 below summarizes the results shown in FIG. 3A.

TABLE 1
Binding affinity to CA-IX

| | Binding affinity to | Binding affinity to |
|---|---|---|
| Clone | monomeric CA-IX (K_{D}) (M) | dimeric CA-IX (K_{D}) (M) |
| CA9-1-V_{H}H | 2.89 × 10⁻⁸ | 3.70 × 10⁻⁸ |
| CA9-2-V_{H}H | 2.34 × 10⁻⁷ | 1.76 × 10⁻⁷ |
| CA9-3-V_{H}H | 3.14 × 10⁻⁷ | 3.41 × 10⁻⁷ |

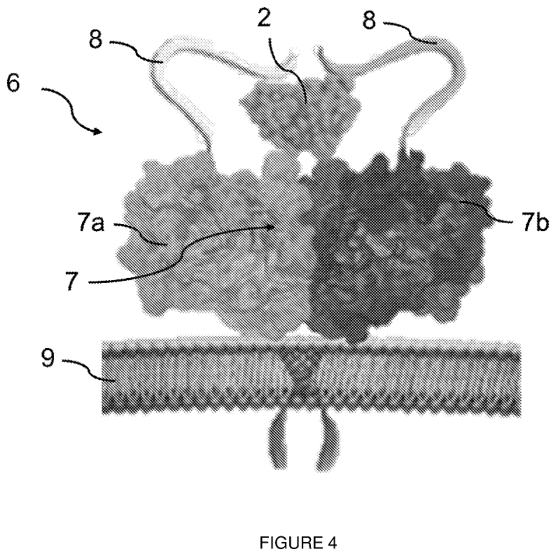

In vitro binding affinity/OCTET. Binding affinity and binding epitope determination of two CA-IX sdAbs in human Fc fused format was carried out using the OCTET platform. The association and dissociation rate of the sdAbs to monomeric parental CA-IX ECD (extracellular domain) and a mutated version lacking the PG domain (APG-CA-IX_{Cys41Ser}) was compared. All antibodies were captured on Anti-human Fc biosensors (AHC) and then dipped and read in wells containing monomeric parental CA-IX or APG-CA-IX_{Cys41Ser}. Collected experimental data was analyzed according to the Global fitting analysis (FIG. 3B). The apparent similar K_{D} of both constructs suggests that the two single domain antibodies sdCA9-1-Fc and sdCA9-2-Fc bind the catalytic domain of CA-IX ECD. The results are summarized in Table 2 below. FIG. 4 is a schematic showing the intracellular/extracellular interface 6 for CA-IX 7. CA-IX 7 is shown in its dimeric form have a first dimer 7a and a second dimer 7b, the PG domain 8, and crosses the lipid bilayer 9 of the cell. The sdCA9-2-Fc 2 is shown binding to the catalytic domain of CA-IX 7.

TABLE 2
Binding affinity and epitope determination

| | | Binding affinity to ΔPG- |
|---|---|---|
| | Binding affinity to hCA- | hCA-IX_{Cys41Ser}-ECD |
| Clone | IX-ECD monomeric (K_{D}) (M) | monomeric (K_{D}) (M) |
| CA9-1-Fc | 1.626 × 10⁻⁸ | 8.184 × 10⁻⁹ |
| CA9-2-Fc | 1.775 × 10⁻⁸ | 1.22075 × 10⁻⁸ |

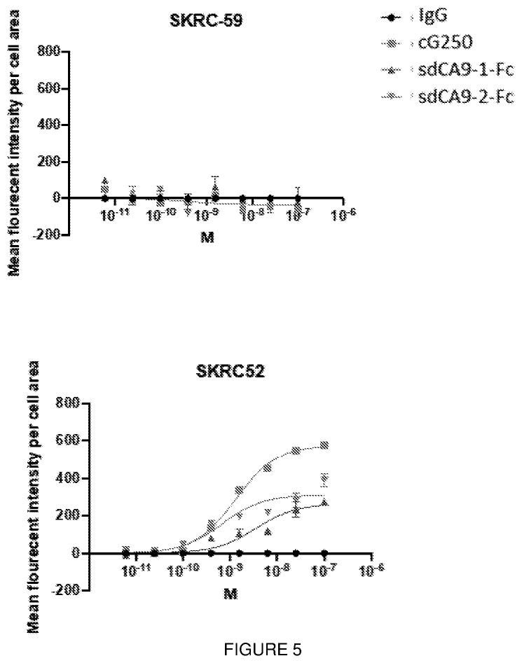

Cell binding. Binding affinity on cells was measured through a cell binding assay performed on a plate format on the non-transfected human clear cells renal carcinoma cell line SKRC59 and SKRC52 cell lines, which have been shown to constitutively express low and high levels of hCA-IX, respectively. These cell lines have been used in the literature for the screening of scFv Abs for binding to hCA-IX (Xu et al. 2010). K_{D} values were determined to be in the low nanomolar range, the clinically used cG250 full sized antibody (FSA) was used as a bench mark. An isotype matching hIgG was used as negative control (FIG. 5). The conditions and results are summarized in Table 3 below.

TABLE 3
Cell binding experiment

| | | | |
|---|---|---|---|
| Control IgG | cG250 | CA9-1-Fc | CA9-2-Fc |

| | |
|---|---|
| One site - | Ambiguous |
| Specific | |
| binding | |

Best-fit values

| | | | | |
|---|---|---|---|---|
| Bₘₐₓ | 0.000 | 574.6 | 264.7 | 311.9 |
| K_{D} | ~9.775 × 10⁻¹⁰ | 1.288 × 10⁻⁹ | 3.493 × 10⁻⁹ | 6.579 × 10⁻¹⁰ |

95% CI (profile likelihood)

| | | | |
|---|---|---|---|
| Bₘₐₓ | 543.9 to 606.2 | 212.8 to 328.5 | 262.6 to 369.4 |
| K_{D} | 9.851 × 10⁻¹⁰ to | 1.071 2.360 × | 2.360 × 10⁻¹⁰ to |
| | 1.681 × 10⁻⁹ | 10⁻⁹ to 9.544 × | 1.942 2.360 × 10⁻⁹ |
| | | 10⁻¹⁰ | |

Binding epitope determination. To identify the binding domain of the antibodies, the stabilization of ECD of CA-IX was evaluated using HDX-MS method. All HDX-MS experiments were performed on hCA-IX catalytic domain monomer construct (PG-CA-IX_{Cys41Ser}) containing a Cys₄₁→Ser mutation, preventing covalent dimer formation, and lacking the proteoglycan (PG) domain (FIGS. 1 and 2). The discovered stabilized epitopes on CA-IX catalytic domain for the two sdAbs (CA9-1-Fc and CA9-2-Fc) were distinct from one another. SdCA9-2-Fc stabilized epitopes at both sides of the catalytic groove including linear epitopes FIG. 2: (a) and alpha helices (b) and (c) on one side of the catalytic groove and (d) on the other side potentially leading to potent inhibition of catalytic domain function, whereas sdCA9-1-Fc binding stabilized a linear region FIG. 1: (a) and alpha helix (c) on one side of the catalytic groove.

TABLE 4
HDX-MS results

| Data set | Control | sdCA9-1-Fc | sdCA9-2-Fc |
|---|---|---|---|
| HDX reaction details | Phosphate buffered saline, | | |
| | pH = 7.0, 20° C. | | |
| HDX time course (min) | 1, 10, 60 | | |
| # of peptides found across all | 78 | | |
| samples | | | |
| Sequence coverage | 82% | | |
| Average peptide length/redundancy | 11.4/34.5 | | |
| Replicates (biological or technical) | 4 (technical) | | |
| Repeatability (average standard | 0.05 | 0.04 | 0.04 |
| deviation, Da) | | | |

| | |
|---|---|
| Significant differences in HDX | Two-state student T-Test |
| | performed at each time point |
| | (>2 SD, p-value 0.05) |

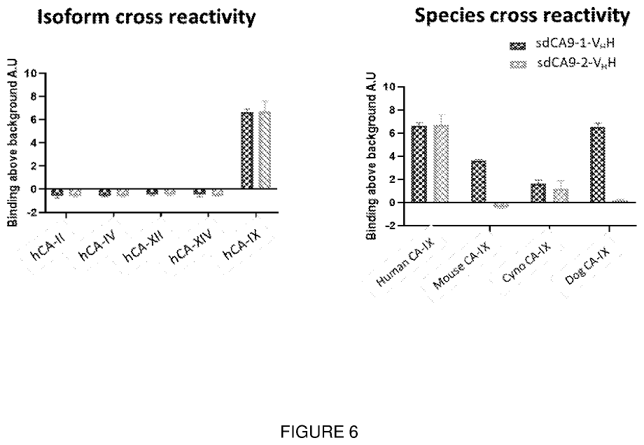

Cross reactivity and CA-IX specificity. Binding specificity and species cross reactivity of two sdAb were tested in an ELISA assay using purified proteins for human, mouse, cynomolgus monkey and dog CA-IX. Both sdAbs show hCA-IX binding selectivity when compared with other carbonic anhydrases CAII, CAIV, CAXII, CAXIV (FIG. 6, upper panel). sdCA9-1-V_{H}H binds mouse, human, cyno and dog CA-IX whereas sdCA9-2-V_{H}H selectively binds human and cyno CA-IX (FIG. 6, lower panel).

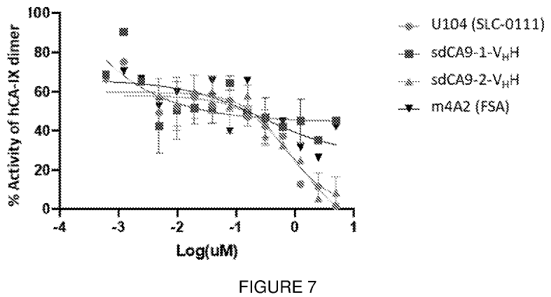

In vitro rhCA-IX enzyme activity inhibition. In order to examine the functional consequence of the binding of the two sdCA9-V_{H}H antibodies to hCA-IX ECD, antibodies were tested in an in vitro enzyme activity assay using purified hCA-IX ECD dimer and 4-Methylumbelliferyl acetate (4Mu-Ac) as substrate. Various antibody concentrations were tested against a fixed concentration of hCA-IX-ECD dimer and calculated IC50 values were compared to those obtained with the CA-IX SMI U104 (AKA SLC-0111) (FIG. 7). Surprisingly, the sdCA9-2-V_{H}H antibody inhibited CA9 dimer at IC₅₀ values that were very similar to the U104 SMI, ranging from 40-48 nM (identical to what was reported in the literature; (Andreucci et al., 2017), whereas hCA-IX inhibition by sdCA9-1-V_{H}H and full size CA-IX m4A2 mAb (U.S. Pat. No. 10,487,153 and WO 2019/204939) is only partially with IC₅₀ values around 100 nM. This unexpected result thus suggests that the sdCA9-2-V_{H}H is very a potent and specific inhibitor of hCA-IX. The results of the inhibition shown in FIG. 7 are also summarized in Table 5 below.

TABLE 5
Results of inhibition assay

| | | | |
|---|---|---|---|
| U104 | sdCA9-1-V_{H}H | sdCA9-2-V_{H}H | m4A2 |

[inhibitor] vs. normalized response - variable slope
Best fit values

| | | | | |
|---|---|---|---|---|
| IC50 | 0.04135 | 0.1112 | 0.04843 | 0.1012 |
| HillSlope | −0.3305 | −0.1194 | −0.2974 | −0.1640 |
| logIC50 | −1.383 | −0.9537 | −1.1315 | −0.9950 |

95% CI (profile likelihood)

| | | | | |
|---|---|---|---|---|
| IC50 | 0.01769 | 0.01900 | 0.02044 | 0.02781 |
| | to 0.08162 | to 1.440 | to 0.09830 | to 0.4698 |
| HillSlope | −0.4441 | −0.2006 | −0.4048 | −0.2450 |
| | to −0.2317 | to −0.04219 | to −0.2041 | to −0.08963 |
| logIC50 | −1.752 | −1.721 | −1.690 | −1.556 |
| | to −1.088 | to 0.1585 | to −1.007 | to −0.381 |

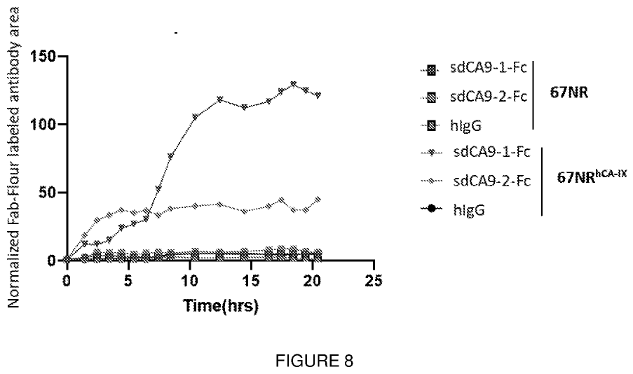

Antibody internalization into cells. The internalization capacity of the two sdCA9 antibodies was evaluated using the parental 67NR mouse mammary carcinoma cell line lacking hCA-IX expression (67NR) and engineered 67NR cells overexpressing hCA-IX (67NR^{hCA-IX}). Fc-fused antibodies were conjugated to the FabFlour pH probe IncuCyte according to manufacturer instructions. The probe displays increased fluorescence upon the decreasing pH during the antibody internalization process. Both conjugated antibodies internalized upon cell surface binding and their rate of internalization was measured by evaluating the fluorescent intensity (FIG. 8) of the FabFluor pH probe over time.

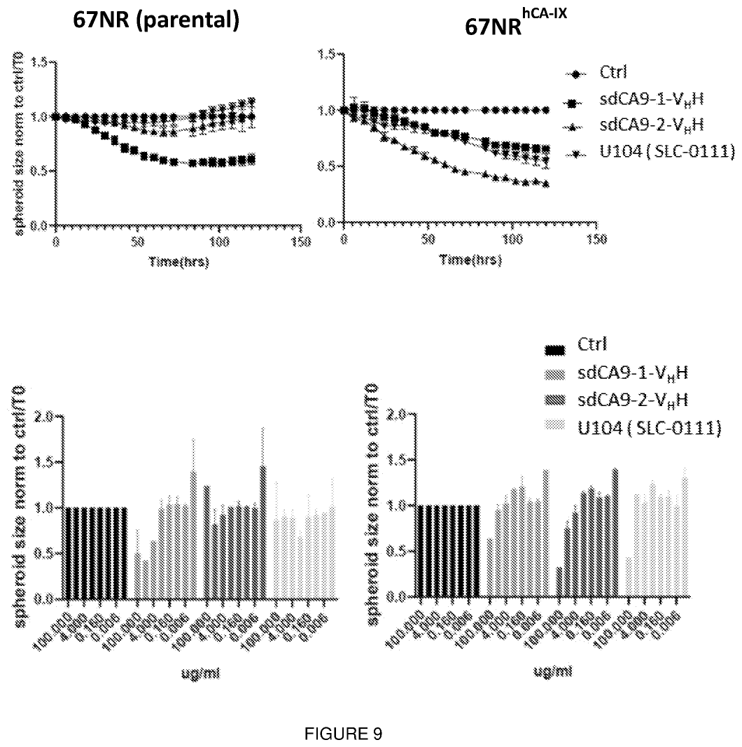

Spheroid growth inhibition. To test if inhibition of the CA-IX enzyme function by the sdCA9 antibodies affected tumor growth, sdCA9-1-V_{H}H and sdCA9-2-V_{H}H were tested on 67NR and 67NR^{hCA-IX} derived spheroids which form spontaneously in low adhesion round-bottom plates. Different concentrations of the antibodies, the U104 small molecule inhibitor and the respective controls were added to the spheroids, after which spheroid growth was monitored for 8 days. While sdCA9-2-V_{H}H inhibited spheroid growth in a dose-dependent manner similar to the U104 SMI, the CA9-1-V_{H}H effect seemed not to be specific as no difference was observed when comparing the 67NR^{hCA-IX} or 67NR derived spheroids (FIG. 9).

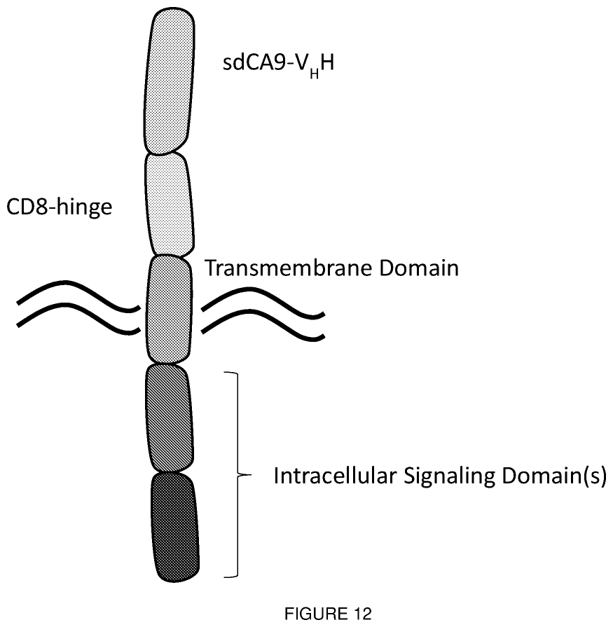

Generation of CAR constructs: After identifying CAIX-binding single domain antibody (sdAb) sequences described above, their activity was tested within the context of chimeric antigen receptor (CAR) molecules which can be used to redirect human T cell responses towards cells bearing specific surface antigens. Thus, using high throughput techniques previously described (Bloemberg 2020) novel CAIX targeted CAR constructs were generated and their target specific T cell activating potential was tested in vitro. The antigen binding domain (ABD) of sdCA9 antibody sequences were transferred to a modular CAR plasmid backbone (e.g., see SEQ ID NO: 28) containing restriction sites that allows efficient recombination wherein the antigen binding domain could be removed and replaced with the novel sdCA9-V_{H}H antigen binding domain (ABD) sequences. Specific CAR design used was as follows: Human CD28 signal peptide (SEQ ID NO: 29), ABD (any one of (e.g., having the amino acid sequence of SEQ ID NO: 1, encoded by the nucleic acid sequence of SEQ ID NO: 21), 2 (e.g., having the amino acid sequence of SEQ ID NO: 5, encoded by the nucleic acid sequence of SEQ ID NO: 22), flexible linker domain (SEQ ID NO: 30), human CD8 hinge domain (SEQ ID NO: 31), human CD28 transmembrane domain (SEQ ID NO: 32), human 4-1 BB signal transduction domain (SEQ ID NO: 33), and human CD3-zeta signal transduction domain (SEQ ID NO: 34). Control constructs were also generated using sequences derived from previously demonstrated CD19-specific CAR sequence. A model of the sdCA9 CAR construct is provided in (FIG. 12).

For some sdAbs, sequence changes were made for testing in the context of CAR molecules. The N-terminal region of sdCA9-2-V_{H}H was changed from QVKLEE to QVQLVESEQ. ID NO: 1), which improved its stability.

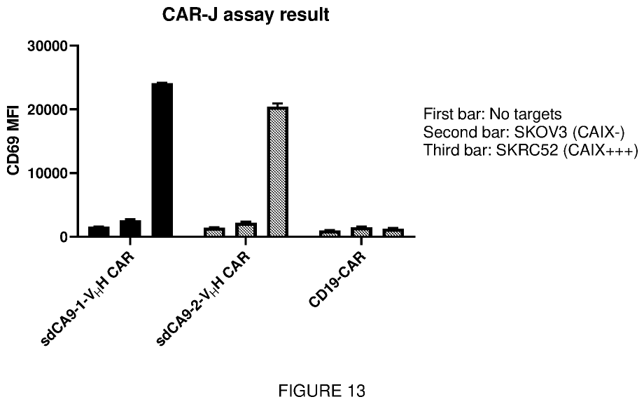

In vitro CAR-Jurkat (CAR-J) assay: CAIX-targeting CAR constructs were then tested for activity in an immortalized human T cell line (Jurkat) as described in Bloemberg et al., 2020. In brief, plasmids were electroporated into Jurkat T cells and allowed to recover for several hours. Jurkat-CAR cells were then mixed with target cell lines exhibiting varying expression levels of CAIX. SKRC52 with high expression of CAIX and SKOV3 without CAIX expression were used as target cells. In order to measure CAR-mediated Jurkat cell activation, expression of CD69 was measured using specific antibody staining and flow cytometry. Using expression of GFP-marker to gate CAR-expressing cells, the level of T cell activation was determined using the CD69-surface marker. CD69 fluorescent intensity signal was elevated in Jurkat cells expressing sdAb CAIX CAR constructs when cells were placed in co-culture with CAIX expressing SKRC52 target cells but not with CAIX-negative SKOV3 cells, a target irrelevant CAR plasmid (CD19-CAR) was used as control (FIG. 13).

Generation of BiTE constructs: Similar to chimeric antigen receptor technology, antigen binding elements can also be linked to CD3-engaging antibody in order generate a soluble molecule that can simultaneously bind T cells and cellular target molecules, resulting in an antigen-specific T cell activation signal. This type of molecule, referred to as a bi-specific T cell engagers (BiTE), is exemplified by Blinatumomab, wherein a single molecule simultaneously engages human CD19 and human CD3; used as a therapy for CD19 expressing B-cell family malignancies. In order to assess whether the CAIX-specific single domain antibodies generated herein could be used in such a bi-specific T cell engager molecule, molecules were generated wherein one end of the molecule was comprised of a CAIX-specific single domain antibody sequence and the other end was comprised of a CD3-engager molecule. These novel bi-specific T cell engagers were then screened for antigen-specific induction of T cell activation.

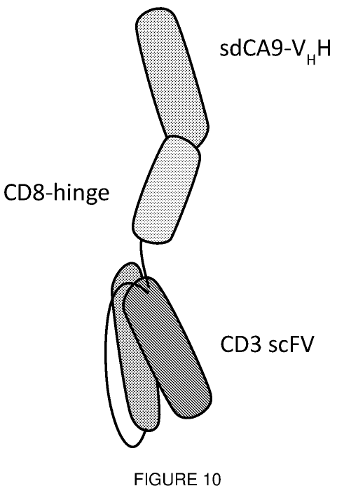

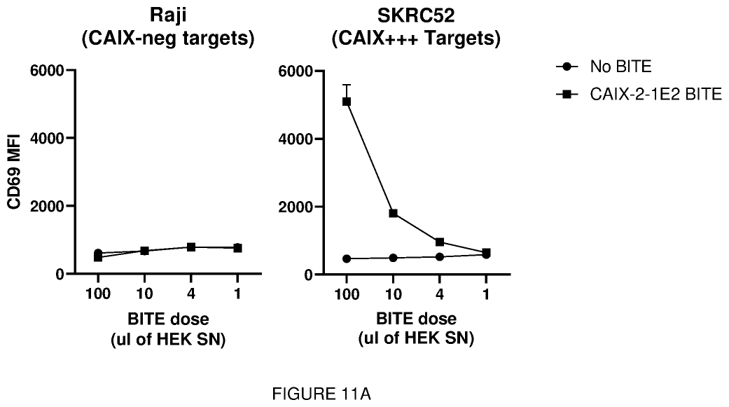

Single domain antibody antigen binding sequences were transferred to a modular bi-specific T cell engager DNA sequence [SEQ. ID NO: 35] within a plasmid backbone; the DNA sequence contains restriction sites to allow efficient recombination wherein the antigen binding domain could be replaced with the CAIX-antigen binding domain (ABD) sequences. Specific bi-specific T cell engager design used was as follows: Human CD28 signal peptide (SEQ ID NO: 29), CAIX sdAb antibody (e.g., having the amino acid sequence of SEQ ID NO: 5, encoded by the nucleic acid sequence of SEQ ID NO: 22), flexible linker domain (SEQ ID NO:30), human CD8 hinge domain (SEQ ID NO: 31), short flexible linker domain (SEQ ID NO: 35), and a CD3-specific single chain variable fragment sequence. A model of CAIX-CD3 bi-specific T cell engager molecules with a hinge/spacer domain is provided (FIG. 10). Constructs were generated using golden gate assembly and confirmed using Sanger sequencing before proceeding to downstream testing. The constructs were then transfected into HEK293T cells using polyethylenimine (PEI) via standard process, supernatant containing the CAIX-2-CD3 BiTE bi specific antibodies were collected for testing.

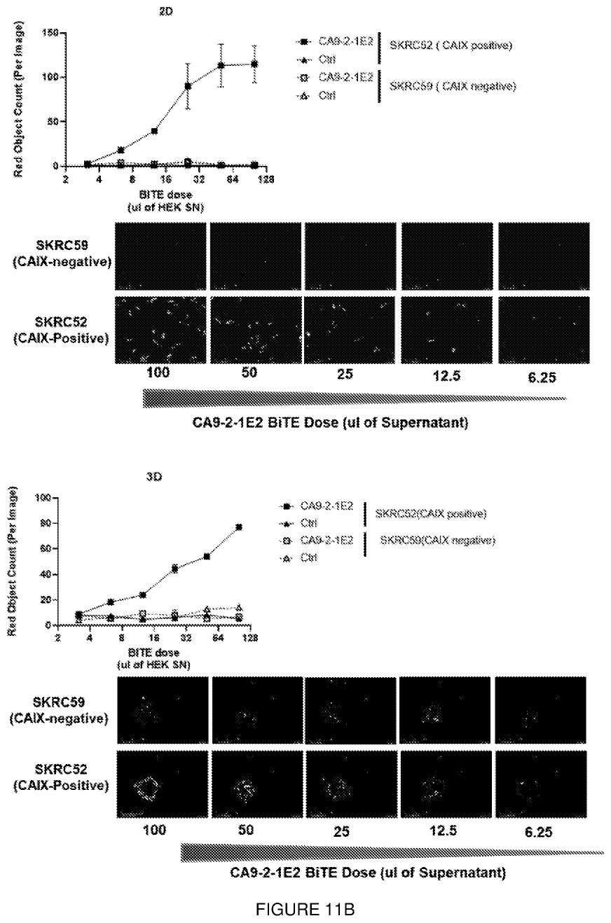

BiTE activity and specificity assay: To evaluate target specific activity of the produced BiTE proteins in vitro supernatant containing CAIX-CD3 bispecific antibodies or control no BiTE were added at varying amounts of directly on Jurkat cells alone or in co-culture with CAIX-positive (SKRC52) or negative (Raji and SKRC59) target cells and incubated under standard conditions overnight. Jurkat cells were then examined for T cell activation using either antibody staining for the human CD69 marker and flow cytometric analysis as described in Bloemberg 2020. Or by using Jurkat-CD69-tdTomato reporter cell line (described in Bloemberg et al., Bloomberg 2021), that reports on CD69 activation downstream T cell activation by expressing td tomato. Number of activated Jurkat cells are then counted by fluorescence imaging and reported as count of td tomato positive cells per image. Results demonstrate that when delivered in solution, a CAIX targeted bi-specific T cell engager can induce target dependent T cell activation in a dose dependant fashion in 2D (FIG. 11A) and 3D (FIG. 11B) cultures.

TABLE 6
Sequence listing

| SEQ | | |
|---|---|---|
| ID | | |
| NO: | Sequence | Description |
| 1 | QVQLVESGGGLVQAGGSLRLSCAAS | Artifical sequence: Amino acid |
| | GFTFDDWAIGWFRQAPGKEREGVSC | sequence for sdCA9-1-V_{H}H (CDR |
| | ISKRHGTTHYADSVKGRFTISSDNAK | sequences underlined) |
| | NTVYLRMNGLKPEDTAVYYCAASSW | |
| | GSCTVATMRDVDRYDYDYWGQGTQ | |
| | VTVSS | |
| 2 | GFTFDDWA | Artifical sequence: Amino acid |
| | | sequence of CDR1 of sdCA9-1- |
| | | V_{H}H |
| 3 | ISKRHGTT | Artifical sequence: Amino acid |
| | | sequence of CDR2 of sdCA9-1- |
| | | V_{H}H |
| 4 | AASSWGSCTVATMRDVDRYDYDY | Artifical sequence: Amino acid |
| | | sequence of CDR3 of sdCA9-1- |
| | | V_{H}H |
| 5 | QVKLEESGGDLVQPGGSLRLSCAAS | Artifical sequence: Amino acid |
| | GFTLDYYAIGWFRQAPGKEREGVSC | sequence for sdCA9-2-V_{H}H (CDR |
| | FSSVDGSTYYADSVKGRFTISRDNAK | sequences undelined) |
| | NMVYLQMNSLKPEDTAIYYCAAELY | |
| | GELGVATVQAMCRLTTPGGDYWGQ | |
| | GTQVTVSS | |
| 6 | GFTLDYYA | Artifical sequence: Amino acid |
| | | sequence of CDR1 of sdCA9-2- |
| | | V_{H}H |
| 7 | FSSVDGST | Artifical sequence: Amino acid |
| | | sequence of CDR2 of sdCA9-2- |
| | | V_{H}H |
| 8 | AAELYGELGVATVQAMCRLTTPGGD | Artifical sequence: Amino acid |
| | Y | sequence CDR3 of sdCA9-2-VHH |
| 9 | QVQLVESGGGLVQAGDSLRLSCAAS | Artificial sequence: a 118 amino |
| | GGTFSRYGMGWFRQAPGKEREFVA | acid sequence for CA9-3-VHH |
| | AISGTGLNTYYMDSVKGRFTISRDNA | (CDR sequences underlined) |
| | ENTVYLQMNSLKPEDTAVYYCARDR | |
| | GGVEYDYWGQGTQVTVSS | |
| 10 | GGTFSRYG | Artifical sequence: Amino acid |
| | | sequence of CDR1 of sdCA9-3- |
| | | V_{H}H |
| 11 | ISGTGLNT | Artifical sequence: Amino acid |
| | | sequence of CDR2 of sdCA9-3- |
| | | V_{H}H |
| 12 | ARDRGGVEYDY | Artifical sequence: Amino acid |
| | | sequence of CDR2 of sdCA9-3- |
| | | V_{H}H |
| 13 | EFATMEFGLSWVFLVAILKGVQC | Artifical sequence: Leader amino |
| | | acid sequence |
| 14 | AEPKSCDKTHTCPPCP | Artifical sequence: Linker amino |
| | | acid sequence |
| 15 | APELLGGPSVFLFPPKPKDTLMISRT | Artifical sequence: hIgG1Fc |
| | PEVTCVVVDVSHEDPEVKFNWYVDG | |
| | VEVHNAKTKPREEQYNSTYRVVSVLT | |
| | VLHQDWLNGKEYKCKVSNKALPAPI | |
| | EKTISKAKGQPREPQVYTLPPSRDEL | |
| | TKNQVSLTCLVKGFYPSDIAVEWESN | |
| | GQPENNYKTTPPVLDSDGSFFLYSKL | |
| | TVDKSRWQQGNVFSCSVMHEALHN | |
| | HYTQKSLSLSPG | |
| 16 | EFATMEFGLSWVFLVAILKGVQC | Artifical sequence: Leader-sdCA9- |
| | QVQLVESGGGLVQAGGSLRLSCAAS | 1-V_{H}H-Linker-hIgG1Fc |
| | GFTFDDWAIGWFRQAPGKEREGVSC | |
| | ISKRHGTTHYADSVKGRFTISSDNAK | |
| | NTVYLRMNGLKPEDTAVYYCAASSW | |
| | GSCTVATMRDVDRYDYDYWGQGTQ | |
| | VTVSSAEPKSCDKTHTCPPCPAPELL | |
| | GGPSVFLFPPKPKDTLMISRTPEVTC | |
| | VVVDVSHEDPEVKFNWYVDGVEVHN | |
| | AKTKPREEQYNSTYRVVSVLTVLHQ | |
| | DWLNGKEYKCKVSNKALPAPIEKTIS | |
| | KAKGQPREPQVYTLPPSRDELTKNQ | |
| | VSLTCLVKGFYPSDIAVEWESNGQPE | |
| | NNYKTTPPVLDSDGSFFLYSKLTVDK | |
| | SRWQQGNVFSCSVMHEALHNHYTQ | |
| | KSLSLSPG | |
| 17 | EFATMEFGLSWVFLVAILKGVQC | Artifical sequence: Leader-sdCA9- |
| | QVKLEESGGDLVQPGGSLRLSCAAS | 2-V_{H}H-Linker-hIgG1Fc |
| | GFTLDYYAIGWFRQAPGKEREGVSC | |
| | FSSVDGSTYYADSVKGRFTISRDNAK | |
| | NMVYLQMNSLKPEDTAIYYCAAELY | |
| | GELGVATVQAMCRLTTPGGDYWGQ | |
| | GTQVTVSS | |
| | AEPKSCDKTHTCPPCPAPELLGGPSV | |
| | FLFPPKPKDTLMISRTPEVTCVVVDV | |
| | SHEDPEVKFNWYVDGVEVHNAKTKP | |
| | REEQYNSTYRVVSVLTVLHQDWLNG | |
| | KEYKCKVSNKALPAPIEKTISKAKGQ | |
| | PREPQVYTLPPSRDELTKNQVSLTCL | |
| | VKGFYPSDIAVEWESNGQPENNYKT | |
| | TPPVLDSDGSFFLYSKLTVDKSRWQ | |
| | QGNVFSCSVMHEALHNHYTQKSLSL | |
| | SPG | |
| 18 | EFATMEFGLSWVFLVAILKGVQC | Artifical sequence: Leader-sdCA9- |
| | QVQLVESGGGLVQAGDSLRLSCAAS | 3-V_{H}H-Linker-hIgG1Fc |
| | GGTFSRYGMGWFRQAPGKEREFVA | |
| | AISGTGLNTYYMDSVKGRFTISRDNA | |
| | ENTVYLQMNSLKPEDTAVYYCARDR | |
| | GGVEYDYWGQGTQVTVSS | |
| | AEPKSCDKTHTCPPCPAPELLGGPSV | |
| | FLFPPKPKDTLMISRTPEVTCVVVDV | |
| | SHEDPEVKFNWYVDGVEVHNAKTKP | |
| | REEQYNSTYRVVSVLTVLHQDWLNG | |
| | KEYKCKVSNKALPAPIEKTISKAKGQ | |
| | PREPQVYTLPPSRDELTKNQVSLTCL | |
| | VKGFYPSDIAVEWESNGQPENNYKT | |
| | TPPVLDSDGSFFLYSKLTVDKSRWQ | |
| | QGNVFSCSVMHEALHNHYTQKSLSL | |
| | SPG | |
| 19 | TATGAAGACACCAGGCCCAGGTGCA | Artifical sequence: Nucleic acid |
| | GCTGGTGGAGTCT | sequence of sense primer V_{H}H |
| | | BbsI |
| 20 | CGCGGGATCCTGAGGAGACGGTGA | Artifical sequence: Nucleic acid |
| | CCTGGGT | sequence of anti-sense primer |
| | | V_{H}H-BamHI |
| 21 | CAGGTACAGCTGGTGGAGTCTGGG | Artifical sequence: Nucleic acid |
| | GGAGGCTTGGTGCAGGCTGGGGGG | sequence of sdCA9-1-V_{H}H |
| | TCTCTGAGACTCTCCTGTGCAGCCT | |
| | CTGGATTCACTTTCGATGATTGGGC | |
| | CATAGGCTGGTTCCGCCAGGCCCC | |
| | AGGGAAGGAGCGTGAGGGGGTCTC | |
| | ATGTATTAGTAAGAGGCATGGTACT | |
| | ACACACTATGCAGACTCCGTGAAGG | |
| | GCCGATTCACCATCTCCAGTGACAA | |
| | CGCCAAGAACACGGTGTATCTGCGA | |
| | ATGAACGGCCTGAAACCTGAGGACA | |
| | CGGCCGTTTATTACTGTGCAGCATC | |
| | CTCCTGGGGATCATGTACTGTAGCG | |
| | ACTATGAGGGACGTTGATAGATATG | |
| | ACTATGACTATTGGGGCCAGGGGAC | |
| | CCAGGTCACCGTCTCCTCA | |
| 22 | CAGGTAAAGCTGGAGGAGTCTGGG | Artifical sequence: Nucleic acid |
| | GGAGACTTGGTGCAGCCTGGGGGG | sequence of sdCA9-2-V_{H}H |
| | TCTCTGAGACTCTCCTGTGCAGCCT | |
| | CTGGATTCACTTTGGATTATTATGCC | |
| | ATAGGCTGGTTCCGCCAGGCCCCA | |
| | GGGAAGGAGCGTGAGGGGGTCTCA | |
| | TGTTTTAGTAGTGTTGATGGTAGCA | |
| | CATACTATGCAGACTCCGTGAAGGG | |
| | CCGATTCACCATCTCCAGAGACAAC | |
| | GCCAAGAACATGGTGTATCTGCAAA | |
| | TGAACAGCCTGAAACCTGAGGACAC | |
| | AGCCATTTATTACTGTGCAGCAGAA | |
| | TTGTACGGGGAGTTGGGAGTGGCT | |
| | ACTGTTCAGGCTATGTGTCGCCTGA | |
| | CTACTCCGGGGGGTGACTACTGGG | |
| | GCCAGGGGACCCAGGTCACCGTCT | |
| | CCTCA | |
| 23 | CAGGTGCAGCTGGTGGAGTCTGGG | Artifical sequence: Nucleic acid |
| | GGAGGATTGGTGCAGGCTGGGGAC | sequence of sdCA9-3-V_{H}H |
| | TCTCTTAGACTCTCCTGTGCAGCCT | |
| | CTGGCGGCACCTTCAGTAGGTATGG | |
| | CATGGGCTGGTTCCGCCAGGCTCC | |
| | AGGGAAGGAGCGTGAGTTTGTCGC | |
| | AGCTATTAGCGGGACTGGTCTGAAT | |
| | ACATACTATATGGACTCCGTGAAGG | |
| | GCCGATTCACCATCTCCAGAGACAA | |
| | CGCCGAGAACACGGTGTATCTGCAA | |
| | ATGAACAGCCTGAAACCTGAGGACA | |
| | CGGCCGTGTATTACTGTGCCAGAGA | |
| | CCGAGGAGGCGTTGAGTATGACTAC | |
| | TGGGGCCAGGGGACCCAGGTCACC | |
| | GTCTCCTCA | |
| 24 | CAGGTACAGCTGGTGGAGTCTGGG | Artifical sequence: Nucleic acid |
| | GGAGGCTTGGTGCAGGCTGGGGGG | sequence of sdCA9-1-linker- |
| | TCTCTGAGACTCTCCTGTGCAGCCT | hIgG1Fc |
| | CTGGATTCACTTTCGATGATTGGGC | |
| | CATAGGCTGGTTCCGCCAGGCCCC | |
| | AGGGAAGGAGCGTGAGGGGGTCTC | |
| | ATGTATTAGTAAGAGGCATGGTACT | |
| | ACACACTATGCAGACTCCGTGAAGG | |
| | GCCGATTCACCATCTCCAGTGACAA | |
| | CGCCAAGAACACGGTGTATCTGCGA | |
| | ATGAACGGCCTGAAACCTGAGGACA | |
| | CGGCCGTTTATTACTGTGCAGCATC | |
| | CTCCTGGGGATCATGTACTGTAGCG | |
| | ACTATGAGGGACGTTGATAGATATG | |
| | ACTATGACTATTGGGGCCAGGGGAC | |
| | CCAGGTCACCGTCTCCTCAGCTGAA | |
| | CCCAAGTCCTGCGACAAGACCCACA | |
| | CCTGTCCCCCCTGCCCTGCCCCTGA | |
| | ACTGCTGGGCGGACCTTCCGTGTTC | |
| | CTGTTCCCCCCAAAGCCTAAGGACA | |
| | CCCTGATGATCTCCCGGACCCCCGA | |
| | AGTGACCTGCGTGGTGGTGGACGT | |
| | GTCCCACGAGGACCCTGAAGTGAA | |
| | GTTCAATTGGTACGTGGACGGCGTG | |
| | GAAGTGCACAACGCCAAGACCAAG | |
| | CCCAGAGAGGAACAGTACAACTCCA | |
| | CCTACCGGGTGGTGTCCGTGCTGA | |
| | CCGTGCTGCACCAGGACTGGCTGA | |
| | ACGGCAAAGAGTACAAGTGCAAGGT | |
| | CTCCAACAAGGCCCTGCCTGCCCC | |
| | CATCGAAAAGACCATCAGCAAGGCC | |
| | AAGGGCCAGCCCCGCGAGCCCCAG | |
| | GTGTACACCCTGCCCCCTAGCCGG | |
| | GACGAGCTGACCAAGAATCAGGTGT | |
| | CCCTGACCTGTCTGGTGAAAGGCTT | |
| | CTACCCCTCCGATATCGCCGTGGAA | |
| | TGGGAGTCCAACGGCCAGCCCGAG | |
| | AACAACTACAAGACCACCCCCCCTG | |
| | TGCTGGACTCCGACGGCTCATTCTT | |
| | CCTGTACTCCAAGCTGACCGTGGAC | |
| | AAGTCCCGGTGGCAGCAGGGCAAC | |
| | GTGTTCTCCTGCTCCGTGATGCACG | |
| | AGGCCCTGCACAACCACTACACCCA | |
| | GAAGTCCCTGTCCCTGAGCCCCGG | |
| | CTGAGGATCC | |
| 25 | CAGGTAAAGCTGGAGGAGTCTGGG | Artifical sequence: Nucleic acid |
| | GGAGACTTGGTGCAGCCTGGGGGG | sequence of sdCA9-2-linker- |
| | TCTCTGAGACTCTCCTGTGCAGCCT | hIgG1Fc |
| | CTGGATTCACTTTGGATTATTATGCC | |
| | ATAGGCTGGTTCCGCCAGGCCCCA | |
| | GGGAAGGAGCGTGAGGGGGTCTCA | |
| | TGTTTTAGTAGTGTTGATGGTAGCA | |
| | CATACTATGCAGACTCCGTGAAGGG | |
| | CCGATTCACCATCTCCAGAGACAAC | |
| | GCCAAGAACATGGTGTATCTGCAAA | |
| | TGAACAGCCTGAAACCTGAGGACAC | |
| | AGCCATTTATTACTGTGCAGCAGAA | |
| | TTGTACGGGGAGTTGGGAGTGGCT | |
| | ACTGTTCAGGCTATGTGTCGCCTGA | |
| | CTACTCCGGGGGGTGACTACTGGG | |
| | GCCAGGGGACCCAGGTCACCGTCT | |
| | CCTCAGCTGAACCCAAGTCCTGCGA | |
| | CAAGACCCACACCTGTCCCCCCTGC | |
| | CCTGCCCCTGAACTGCTGGGCGGA | |
| | CCTTCCGTGTTCCTGTTCCCCCCAA | |
| | AGCCTAAGGACACCCTGATGATCTC | |
| | CCGGACCCCCGAAGTGACCTGCGT | |
| | GGTGGTGGACGTGTCCCACGAGGA | |
| | CCCTGAAGTGAAGTTCAATTGGTAC | |
| | GTGGACGGCGTGGAAGTGCACAAC | |
| | GCCAAGACCAAGCCCAGAGAGGAA | |
| | CAGTACAACTCCACCTACCGGGTGG | |
| | TGTCCGTGCTGACCGTGCTGCACCA | |
| | GGACTGGCTGAACGGCAAAGAGTA | |
| | CAAGTGCAAGGTCTCCAACAAGGCC | |
| | CTGCCTGCCCCCATCGAAAAGACCA | |
| | TCAGCAAGGCCAAGGGCCAGCCCC | |
| | GCGAGCCCCAGGTGTACACCCTGC | |
| | CCCCTAGCCGGGACGAGCTGACCA | |
| | AGAATCAGGTGTCCCTGACCTGTCT | |
| | GGTGAAAGGCTTCTACCCCTCCGAT | |
| | ATCGCCGTGGAATGGGAGTCCAAC | |
| | GGCCAGCCCGAGAACAACTACAAG | |
| | ACCACCCCCCCTGTGCTGGACTCC | |
| | GACGGCTCATTCTTCCTGTACTCCA | |
| | AGCTGACCGTGGACAAGTCCCGGT | |
| | GGCAGCAGGGCAACGTGTTCTCCT | |
| | GCTCCGTGATGCACGAGGCCCTGC | |
| | ACAACCACTACACCCAGAAGTCCCT | |
| | GTCCCTGAGCCCCGGCTGAGGATC | |
| | C | |
| 26 | CAGGTGCAGCTGGTGGAGTCTGGG | Artifical sequence: Nucleic acid |
| | GGAGGATTGGTGCAGGCTGGGGAC | sequence of sdCA9-3-linker- |
| | TCTCTTAGACTCTCCTGTGCAGCCT | hIgG1Fc |
| | CTGGCGGCACCTTCAGTAGGTATGG | |
| | CATGGGCTGGTTCCGCCAGGCTCC | |
| | AGGGAAGGAGCGTGAGTTTGTCGC | |
| | AGCTATTAGCGGGACTGGTCTGAAT | |
| | ACATACTATATGGACTCCGTGAAGG | |
| | GCCGATTCACCATCTCCAGAGACAA | |
| | CGCCGAGAACACGGTGTATCTGCAA | |
| | ATGAACAGCCTGAAACCTGAGGACA | |
| | CGGCCGTGTATTACTGTGCCAGAGA | |
| | CCGAGGAGGCGTTGAGTATGACTAC | |
| | TGGGGCCAGGGGACCCAGGTCACC | |
| | GTCTCCTCA | |
| | GCTGAACCCAAGTCCTGCGACAAGA | |
| | CCCACACCTGTCCCCCCTGCCCTGC | |
| | CCCTGAACTGCTGGGCGGACCTTC | |
| | CGTGTTCCTGTTCCCCCCAAAGCCT | |
| | AAGGACACCCTGATGATCTCCCGGA | |
| | CCCCCGAAGTGACCTGCGTGGTGG | |
| | TGGACGTGTCCCACGAGGACCCTG | |
| | AAGTGAAGTTCAATTGGTACGTGGA | |
| | CGGCGTGGAAGTGCACAACGCCAA | |
| | GACCAAGCCCAGAGAGGAACAGTA | |
| | CAACTCCACCTACCGGGTGGTGTCC | |
| | GTGCTGACCGTGCTGCACCAGGAC | |
| | TGGCTGAACGGCAAAGAGTACAAGT | |
| | GCAAGGTCTCCAACAAGGCCCTGC | |
| | CTGCCCCCATCGAAAAGACCATCAG | |
| | CAAGGCCAAGGGCCAGCCCCGCGA | |
| | GCCCCAGGTGTACACCCTGCCCCC | |
| | TAGCCGGGACGAGCTGACCAAGAA | |
| | TCAGGTGTCCCTGACCTGTCTGGTG | |
| | AAAGGCTTCTACCCCTCCGATATCG | |
| | CCGTGGAATGGGAGTCCAACGGCC | |
| | AGCCCGAGAACAACTACAAGACCAC | |
| | CCCCCCTGTGCTGGACTCCGACGG | |
| | CTCATTCTTCCTGTACTCCAAGCTGA | |
| | CCGTGGACAAGTCCCGGTGGCAGC | |
| | AGGGCAACGTGTTCTCCTGCTCCGT | |
| | GATGCACGAGGCCCTGCACAACCA | |
| | CTACACCCAGAAGTCCCTGTCCCTG | |
| | AGCCCCGGCTGAGGATCC | |
| 27 | GCCCCTGAACTGCTGGGGGGACCT | Artifical sequence: Nucleic acid |
| | TCCGTGTTCCTGTTCCCCCCAAAGC | sequence encoding hIgG1Fc |
| | CTAAGGACACCCTGATGATCTCCCG | (SEQ ID NO: 15) |
| | GACCCCCGAAGTGACCTGCGTGGT | |
| | GGTGGACGTGTCCCACGAGGACCC | |
| | TGAAGTGAAGTTCAATTGGTACGTG | |
| | GACGGCGTGGAAGTGCACAACGCC | |
| | AAGACCAAGCCCAGAGAGGAACAG | |
| | TACAACTCCACCTACCGGGTGGTGT | |
| | CCGTGCTGACCGTGCTGCACCAGG | |
| | ACTGGCTGAACGGCAAAGAGTACAA | |
| | GTGCAAGGTCTCCAACAAGGCCCTG | |
| | CCTGCCCCCATCGAAAAGACCATCA | |
| | GCAAGGCCAAGGGCCAGCCCCGCG | |
| | AGCCCCAGGTGTACACCCTGCCCC | |
| | CTAGCCGGGACGAGCTGACCAAGA | |
| | ATCAGGTGTCCCTGACCTGTCTGGT | |
| | GAAAGGCTTCTACCCCTCCGATATC | |
| | GCCGTGGAATGGGAGTCCAACGGC | |
| | CAGCCCGAGAACAACTACAAGACCA | |
| | CCCCCCCTGTGCTGGACTCCGACG | |
| | GCTCATTCTTCCTGTACTCCAAGCT | |
| | GACCGTGGACAAGTCCCGGTGGCA | |
| | GCAGGGCAACGTGTTCTCCTGCTCC | |
| | GTGATGCACGAGGCCCTGCACAAC | |
| | CACTACACCCAGAAGTCCCTGTCCC | |
| | TGAGCCCCGGCTGAGGATCC | |
| 28 | atgctcaggctgctcttggctctcaacttatt | Artifical sequence: Nucleic acid |
| | cccttcaattcaagtaacaggaggGTCTTC | sequence encoding CAR modular |
| | . . . [ABD_sequence ] . . . | construct (SEQ ID NO: 28) |
| | GAAGACttCCTTTGcGAGACGacGGTGGCGGG | |
| | GGATCAGGTGGTGGAGGTAGCGGGGGAGGGGG | |
| | CTCAGGCGGTACAACTACGCCTGCACCTCGCC | |
| | CACCGACCCCAGCACCAACCATCGCTTCACAG | |
| | CCTTTGAGCCTGCGACCAGAGGCATGTCGCCC | |
| | TGCTGCGGGCGGTGCCGTTCATACTCGCGGAC | |
| | TTGATTTTGCGTGTGACgtCGTCTCgccttct | |
| | aagcccttttgggtgctggtggtggttggtgg | |
| | agtcctggcttgctatagcttgctagtaacag | |
| | tggcctttattattttctgggtgaggaaacgg | |
| | ggcagaaagaaactcctgtatatattcaaaca | |
| | accatttatgCgaccagtacaaactactcaag | |
| | aggaagatggctgtagctgccgatttccagaa | |
| | gaagaagaaggaggatgtgaactgctgagagt | |
| | gaagttcagcaggagcgcagacgcccccgcgt | |
| | accagcagggccagaaccagctctataacgag | |
| | ctcaatctaggacgaagagaggagtacgatgt | |
| | tttggacaagCgacgtggccgggaccctgaga | |
| | tggggggaaagccgcagagaaggaagaaccct | |
| | caggaaggcctgtacaatgaactgcagaaaga | |
| | taagatggcggaggcctacagtgagattggga | |
| | tgaaaggcgagcgccggaggggcaaggggcac | |
| | gatggcctttaccagggActcagtacagccac | |
| | caaggacacctacgacgcccttcacatgcagg | |
| | ccctgccccctcgcGCTAGCGCCACGAACTTC | |
| | TCTCTGTTAAAGCAAGCAGGCGACGTGGAAGA | |
| | AAACCCCGGTCCCATGGTGAGCAAGGGCGAGG | |
| | AGGACAACATGGCCAGCCTGCCCGCCACCCAC | |
| | GAGCTGCACATCTTCGGCAGCATCAACGGCGT | |
| | GGACTTCGACATGGTGGGCCAGGGCACCGGCA | |
| | ACCCCAACGACGGCTACGAGGAGCTGAACCTG | |
| | AAGAGCACCAAGGGCGACCTGCAGTTCAGCCC | |
| | CTGGATtCTGGTGCCCCACATCGGCTACGGCT | |
| | TCCACCAGTACCTGCCCTACCCCGACGGCATG | |
| | AGCCCCTTCCAGGCCGCCATGGTGGACGGCAG | |
| | CGGCTACCAGGTGCACAGGACCATGCAGTTCG | |
| | AGGACGGCGCCAGCCTGACCGTGAACTACAGG | |
| | TACACCTACGAGGGCAGCCACATCAAGGGCGA | |
| | GGCCCAGGTGAAGGGCACCGGCTTCCCCGCCG | |
| | ACGGCCCCGTGATGACCAACAGCCTGACCGCC | |
| | GCCGACTGGTGCAGGAGCAAAAAGACCTACCC | |
| | CAACGACAAGACCATCATCAGCACCTTCAAGT | |
| | GGAGCTACACCACCGGCAACGGCAAGAGGTAC | |
| | AGGAGCACCGCCAGGACCACCTACACCTTCGC | |
| | CAAGCCCATGGCCGCCAACTACCTGAAGAACC | |
| | AGCCCATGTACGTGTTCAGAAAGACCGAGCTG | |
| | AAGCACAGCAAGACCGAGCTGAACTTCAAGGA | |
| | GTGGCAGAAGGCCTTCACCGACGTGATGGGCA | |
| | TGGACGAGCTGTACAAGCCCAAGAAGAAGAGG | |
| | AAGGTGGAGGACCCCCCCGCCGCCAAGAGGGT | |
| | GAAGCTGGACTaa | |
| 29 | MLRLLLALNLFPSIQVTG | Human CD28 Signal Peptide |
| 30 | GGGGSGGGGSGGGGSGG | Synthetic Flexible Linker Domain |
| | | (exemplary) |
| 31 | TTTPAPRPPTPAPTIASQPLSLRPEACRPAAG | Human CD8 Hinge Domain |
| | GAVHTRGLDFACD | |
| 32 | PSKPFWVLVVVGGVLACYSLLVTVAFIIFWVR | Human CD28 Transmembrane |
| | | Domain |
| 33 | KRGRKKLLYIFKQPFMRPVQTTQEEDGCSCRE | Human 4-1BB Costimulatory Domain |
| | PEEEEGGCEL | |
| 34 | LRVKFSRSADAPAYQQGQNQLYNELNLGRREE | Human CD3zeta Signaling Domain |
| | YDVLDKRRGRDPEMGGKPQRRKNPQEGLYNEL | |
| | QKDKMAEAYSEIGMKGERRRGKGHDGLYQGLS | |
| | TATKDTYDALHMQALPPR | |
| 35 | GGGGS | Short flexible linker amino acid |
| | | sequence |
| 36 | ATGGAGTTTGGGCTGAGCTGGGTTTTCCTCGT | Artifical sequence: Nucleic acid |
| | TGCTCTTTTTAGAGGTGTCCAGTGTACAGGAG | sequence Bispecific T cell engager |
| | GGTCTTCG. . . [ABD_sequence ] | modular construct DNA sequence |
| | . . .gaagactt | (SEQ ID NO: 36) |
| | ccttggaggaggcggaagt [anti-CD3 | |
| | Variable Heavy Chain sequence] | |
| | ggcggtggtgggtcaggcggcggtgggagcgg | |
| | aggaggtggaagc [anti-CD3 Variable | |
| | Light Chain sequence] TAG | |
| 37 | MLRLLLALNLFPSIQVTGQVQLVESGGGLVQA | Artifical sequence: sdCA9-1-V_{H}H |
| | GGSLRLSCAASGFTFDDWAIGWFRQAPGKERE | CAR amino acid sequence |
| | GVSCISKRHGTTHYADSVKGRETISSDNAKNT | Exemplary sequence for a single |
| | VYLRMNGLKPEDTAVYYCAASSWGSCTVATMR | binder CAR construct |
| | DVDRYDYDYWGQGTQVTVSSPSGGGGPSTTTP | |
| | APRPPTPAPTIASQPLSLRPEACRPAAGGAVH | |
| | TRGLDFACDPSKPFWVLVVVGGVLACYSLLVT | |
| | VAFIIFWVRKRGRKKLLYIFKQPFMRPVQTTQ | |
| | EEDGCSCRFPEEEEGGCELLRVKFSRSADAPA | |
| | YQQGQNQLYNELNLGRREEYDVLDKRRGRDPE | |
| | MGGKPQRRKNPQEGLYNELQKDKMAEAYSEIG | |
| | MKGERRRGKGHDGLYQGLSTATKDTYDALHMQ | sdAb sequence in bold |
| | ALPPR | |
| 38 | MLRLLLALNLFPSIQVTGQVKLEESGGDLVQP | Artifical sequence: sdCA9-2-V_{H}H |
| | GGSLRLSCAASGFTLDYYAIGWFRQAPGKERE | CAR amino acid sequence |
| | GVSCFSSVDGSTYYADSVKGRFTISRDNAKNM | sdAb sequence in bold |
| | VYLQMNSLKPEDTAIYYCAAELYGELGVATVQ | |
| | AMCRLTTPGGDYWGQGTQVTVSSPSGGGGPST | |
| | TTPAPRPPTPAPTIASQPLSLRPEACRPAAGG | |
| | AVHTRGLDFACDPSKPFWVLVVVGGVLACYSL | |
| | LVTVAFIIFWVRKRGRKKLLYIFKQPFMRPVQ | |
| | TTQEEDGCCRFPEEEEGGCELLRVKFSRSAD | |
| | APAYQQGQNQLYNELNLGRREEYDVLDKRRGR | |
| | DPEMGGKPQRRKNPQEGLYNELQKDKMAEAYS | |
| | EIGMKGERRRGKGHDGLYQGLSTATKDTYDAL | |
| | HMQALPPR | |
| 39 | MEFGLSWVFLVALFRGVQCTGQVKLEESGGDL | Artifical sequence: |
| | VQPGGSLRLSCAASGFTLDYYAIGWFRQAPGK | sdCAIX2-CD3scFv amino acid |
| | EREGVSCFSSVDGSTYYADSVKGRFTISRDNA | sequence |
| | KNMVYLQMNSLKPEDTAIYYCAAELYGELGVA | Exemplary sequence for a CAIX |
| | TVQAMCRLTTPGGDYWGQGTQVTVSSGGGGSG | bispecific immune engager |
| | GGGSGGGGSGGTTTPAPRPPTPAPTIASQPLS | construct |
| | LRPEACRPAAGGAVHTRGLDFACD[CD3- | sdAb in bold |
| | specific_scFv_Sequence]HHHHHH* | |

As seen therefore, the examples described above and illustrated are intended to be exemplary only. The scope is indicated by the appended claims.

### REFERENCES

Andreucci, Elena, Silvia Peppicelli, Fabrizio Carta, Giulia Brisotto, Eva Biscontin, Jessica Ruzzolini, Francesca Bianchini, Alessio Biagioni, Claudiu T. Supuran, and Lido Calorini. 2017. “Carbonic Anhydrase IX Inhibition Affects Viability of Cancer Cells Adapted to Extracellular Acidosis.” Journal of Molecular Medicine 95 (12): 1341-53. https://doi.org/10.1007/s00109-017-1590-9.
Arbabi-Ghahroudi, Mehdi, Roger MacKenzie, and Jamshid Tanha. 2009. “Selection of Non-Aggregating VH Binders from Synthetic VH Phage-Display Libraries.” Methods in Molecular Biology (Clifton, N.J.). https://doi.org/10.1007/978-1-59745-554-1 10.
Patrick J. Doyle, Mehdi Arbabi-Ghahroudi, Nathalie Gaudette, Gordon Furzer, Marc E. Savard, Steve Gleddie, Michael D. McLean, C. Roger Mackenzie, J. Christopher Hall, Cloning, expression, and characterization of a single-domain antibody fragment with affinity for 15-acetyl-deoxynivalenol, Molecular Immunology, Volume 45, Issue 14, 2008, https://doi.org/10.1016/j.molimm.2008.06.005
WILBUR K M, ANDERSON N G. Electrometric and colorimetric determination of carbonic anhydrase. J Biol Chem. 1948 October; 176(1):147-54. PMID: 18886152.
U.S. patent Ser. No. 10/487,153
WO 2019/204939
Arbabi Ghahroudi, M., Desmyter, A., Wyns, L., Hamers, R., & Muyldermans, S. (1997). Selection and identification of single domain antibody fragments from camel heavy-chain antibodies. FEBS Letters, 414(3), 521-526. https://doi.org/10.1016/s0014-5793(97)01062-4
Nocentini, A., & Supuran, C. T. (2018). Carbonic anhydrase inhibitors as antitumor/antimetastatic agents: a patent review (2008-2018). Expert Opinion on Therapeutic Patents, 28(10), 729-740. https://doi.org/10.1080/13543776.2018.1508453
Zhang, J., Tanha, J., Hirama, T., Khieu, N. H., To, R., Tong-Sevinc, H., Stone, E., Brisson, J. R., & MacKenzie, C. R. (2004). Pentamerization of single-domain antibodies from phage libraries: a novel strategy for the rapid generation of high-avidity antibody reagents. Journal of Molecular Biology, 335(1), 49-56. https://doi.org/10.1016/j.jmb.2003.09.034
Iqbal, U., Trojahn, U., Albaghdadi, H., Zhang, J., O'Connor-McCourt, M., Stanimirovic, D., Tomanek, B., Sutherland, G., & Abulrob, A. (2010). Kinetic analysis of novel mono- and multivalent VHH-fragments and their application for molecular imaging of brain tumours. British Journal of Pharmacology, 160(4), 1016-1028. https://doi.org/10.1111/j.1476-5381.2010.00742.x
Bell, A., Wang, Z. J., Arbabi-Ghahroudi, M., Chang, T. A., Durocher, Y., Trojahn, U., Baardsnes, J., Jaramillo, M. L., Li, S., Baral, T. N., O'Connor-McCourt, M., MacKenzie, R., & Zhang, J. (2010). Differential tumor-targeting abilities of three single-domain antibody formats. Cancer Letters, 289(1), 81-90. https://doi.org/https://doi.org/10.1016/j.canlet.2009.08.003
Bloemberg, Darin et al. A High-Throughput Method for Characterizing Novel Chimeric Antigen Receptors in Jurkat Cells. Molecular Therapy—Methods & Clinical Development. 2020, 16:238-254
Bloemberg, D., Sosa-Miranda, C. D., Nguyen, T., Weeratna, R. D., & McComb, S. (2021). Self-Cutting and Integrating CRISPR Plasmids Enable Targeted Genomic Integration of Genetic Payloads for Rapid Cell Engineering. The CRISPR Journal, 4(1), 104-119. https://doi.org/10.1089/crispr.2020.0090
Ridgway, J. B., Presta, L. G., & Carter, P. (1996). “Knobs-into-holes” engineering of antibody CH3 domains for heavy chain heterodimerization. Protein Engineering, 9(7), 617-621. https://doi.org/10.1093/protein/9.7.617
de Kruif, J., & Logtenberg, T. (1996). Leucine zipper dimerized bivalent and bispecific scFv antibodies from a semi-synthetic antibody phage display library. The Journal of Biological Chemistry, 271(13), 7630-7634. https://doi.org/10.1074/jbc.271.13.7630
Nielsen, U. B., Adams, G. P., Weiner, L. M., & Marks, J. D. (2000). Targeting of bivalent anti-ErbB2 diabody antibody fragments to tumor cells is independent of the intrinsic antibody affinity. Cancer Research, 60(22), 6434-6440.
Zhu, X., Wang, L., Liu, R., Flutter, B., Li, S., Ding, J., Tao, H., Liu, C., Sun, M., & Gao, B. (2010). COMBODY: one-domain antibody multimer with improved avidity. Immunology and Cell Biology, 88(6), 667-675. https://doi.org/10.1038/icb.2010.21
Merritt, E. A., Sarfaty, S., Pizza, M., Domenighini, M., Rappuoli, R., & Hol, W. G. J. (1995). Mutation of a buried residue causes loss of activity but no conformational change in the heat-labile enterotoxin of Escherichia coli. Nature Structural Biology, 2(4), 269-272. https://doi.org/10.1038/nsb0495-269
Nicaise, M., Valerio-Lepiniec, M., Minard, P., & Desmadril, M. (2004). Affinity transfer by CDR grafting on a nonimmunoglobulin scaffold. Protein Science: A Publication of the Protein Society, 13(7), 1882-1891. https://doi.org/10.1110/ps.03540504
Lefranc, M.-P., Pommié, C., Ruiz, M., Giudicelli, V., Foulquier, E., Truong, L., Thouvenin-Contet, V., & Lefranc, G. (2003). IMGT unique numbering for immunoglobulin and T cell receptor variable domains and Ig superfamily V-like domains. Developmental and Comparative Immunology, 27(1), 55-77. https://doi.org/10.1016/s0145-305x(02)00039-3
Kim, H.-J., McCoy, M. R., Majkova, Z., Dechant, J. E., Gee, S. J., Tabares-da Rosa, S., Gonzalez-Sapienza, G. G., & Hammock, B. D. (2012). Isolation of Alpaca Anti-Hapten Heavy Chain Single Domain Antibodies for Development of Sensitive Immunoassay. Analytical Chemistry, 84(2), 1165-1171. https://doi.org/10.1021/ac2030255
Hussack, G., Arbabi-Ghahroudi, M., van Faassen, H., Songer, J. G., Ng, K. K.-S., MacKenzie, R., & Tanha, J. (2011). Neutralization of Clostridium difficile toxin A with single-domain antibodies targeting the cell receptor binding domain. The Journal of Biological Chemistry, 286(11), 8961-8976. https://doi.org/10.1074/jbc.M110.198754
Davies, J., & Riechmann, L. (1995). Antibody VH Domains as Small Recognition Units. Bio/Technology, 13(5), 475-479. https://doi.org/10.1038/nbt0595-475
Dumoulin, M., Conrath, K., Van Meirhaeghe, A., Meersman, F., Heremans, K., Frenken, L. G. J., Muyldermans, S., Wyns, L., & Matagne, A. (2002). Single-domain antibody fragments with high conformational stability. Protein Science: A Publication of the Protein Society, 11(3), 500-515. https://doi.org/10.1110/ps.34602
To, R., Hirama, T., Arbabi-Ghahroudi, M., MacKenzie, R., Wang, P., Xu, P., Ni, F., & Tanha, J. (2005). Isolation of monomeric human V(H)s by a phage selection. The Journal of Biological Chemistry, 280(50), 41395-41403. https://doi.org/10.1074/jbc.M509900200
Jespers, L., Schon, O., Famm, K., & Winter, G. (2004). Aggregation-resistant domain antibodies selected on phage by heat denaturation. In Nature biotechnology (Vol. 22, Issue 9, pp. 1161-1165). https://doi.org/10.1038/nbt1000
Nuttall, S. D., Krishnan, U. V, Doughty, L., Pearson, K., Ryan, M. T., Hoogenraad, N. J., Hattarki, M., Carmichael, J. A., Irving, R. A., & Hudson, P. J. (2003). Isolation and characterization of an IgNAR variable domain specific for the human mitochondrial translocase receptor Tom70. European Journal of Biochemistry, 270(17), 3543-3554. https://doi.org/10.1046/j.1432-1033.2003.03737.x
Hamers-Casterman, C., Atarhouch, T., Muyldermans, S., Robinson, G., Hammers, C., Songa, E. B., Bendahman, N., & Hammers, R. (1993). Naturally occurring antibodies devoid of light chains. Nature, 363(6428), 446-448. https://doi.org/10.1038/363446a0

1. A single domain antibody that specifically binds to an epitope in a catalytic domain of carbonic anhydrase IX (CA-IX), wherein the single domain antibody has a dissociation constant (K_{D}) of 1×10⁻⁷ or lower for a monomeric form of CA-IX and/or a dimeric form of CA-IX.

2. The single domain antibody according to claim 1, wherein the single domain antibody:
has a K_{D} of 9×10⁻¹ or lower for a CA-IX fragment lacking a proteoglycan-like (PG) domain;
is capable of reducing the biological activity of CA-IX; and/or
is capable of being internalized by a cell expressing CA-IX.

3-5. (canceled)

6. The single domain antibody of claim 1, wherein the single domain antibody lacks the ability to specifically bind to at least one of: carbonic anhydrase II (CA-II), carbonic anhydrase IV (CA-IV), carbonic anhydrase XII (CA-XII), carbonic anhydrase XIV (CA-XIV), and a proteoglycan-like (PG) domain of CA-IX.

7. The single domain antibody of claim 1 comprising:
a first complementary determining region (CDR) having the amino acid sequence of SEQ ID NO: 2, a variant of SEQ ID NO: 2 or a fragment of SEQ ID NO: 2; SEQ ID NO: 6, a variant of SEQ ID NO: 6 or a fragment of SEQ ID NO: 6; or SEQ ID NO: 10, a variant of SEQ ID NO: 10 or a fragment of SEQ ID NO: 10;
a second CDR having the amino acid sequence of sequence of SEQ ID NO: 3, a variant of SEQ ID NO: 3 or a fragment of SEQ ID NO: 3; SEQ ID NO: 7, a variant of SEQ ID NO: 7 or a fragment of SEQ ID NO: 7; or SEQ ID NO: 11, a variant of SEQ ID NO: 11 or a fragment of SEQ ID NO: 11; and/or
a third CDR having the amino acid sequence of SEQ ID NO: 4, a variant of SEQ ID NO: 4 or a fragment of SEQ ID NO: 4; SEQ ID NO: 8, a variant of SEQ ID NO: 8 or a fragment of SEQ ID NO: 8; or SEQ ID NO: 12, a variant of SEQ ID NO: 12 or a fragment of SEQ ID NO: 12.

8. The single domain antibody of claim 7 comprising:
at least one of the first CDR having the amino acid sequence of SEQ ID NO: 2, a variant of SEQ ID NO: 2 or a fragment of SEQ ID NO: 2; the second CDR having the amino acid sequence of SEQ ID NO: 3, a variant of SEQ ID NO: 3 or a fragment of SEQ ID NO: 3 or the third CDR having the amino acid sequence of SEQ ID NO: 4, a variant of SEQ ID NO: 4 or a fragment of SEQ ID NO: 4;
at least one of the first CDR having the amino acid sequence of SEQ ID NO: 6, a variant of SEQ ID NO: 6 or a fragment of SEQ ID NO: 6; the second CDR having the amino acid sequence of SEQ ID NO: 7, a variant of SEQ ID NO: 7 or a fragment of SEQ ID NO: 7; or the third CDR having the amino acid sequence of SEQ ID NO: 8, a variant of SEQ ID NO: 8 or a fragment of SEQ ID NO: 8; or
at least one of the first CDR having the amino acid sequence of SEQ ID NO: 10, a variant of SEQ ID NO: 10 or a fragment of SEQ ID NO: 10; the second CDR having the amino acid sequence of SEQ ID NO: 11, a variant of SEQ ID NO: 11 or a fragment of SEQ ID NO: 11; or the third CDR having the amino acid sequence of SEQ ID NO: 12, a variant of SEQ ID NO: 12 or a fragment of SEQ ID NO: 12.

9. The single domain antibody of claim 8 having:
the amino acid sequence of SEQ ID NO: 1, a variant of SEQ ID NO: 1 or a fragment of SEQ ID NO: 1;
the amino acid sequence of SEQ ID NO: 5, a variant of SEQ ID NO: 5 or a fragment of SEQ ID NO: 5, a variant of SEQ ID NO: 5 or a fragment of SEQ ID NO: 5; or
the amino acid sequence of SEQ ID NO: 9, a variant of SEQ ID NO: 9 or a fragment of SEQ ID NO: 9.

10-15. (canceled)

16. The single domain antibody of claim 1, wherein the single domain antibody is associated with a toxic payload or a detectable label.

17. A multivalent antibody comprising at least single domain antibody of claim 1.

18. (canceled)

19. A chimeric polypeptide comprising (i) at least one single domain antibody of claim 1 and (ii) a carrier polypeptide.

20. The chimeric polypeptide of claim 19, wherein the carrier polypeptide is an Fc, an antibody, an antibody fragment, a serum protein, a chimeric antigen receptor (CAR) construct or a bispecific T cell engager (BiTE) construct.

21. The chimeric polypeptide of claim 20, wherein the Fc has the amino acid sequence of SEQ ID NO: 15, is a variant of the amino acid sequence of SEQ ID NO: 15 or is a fragment of the amino acid sequence of SEQ ID NO: 15.

22. (canceled)

23. The chimeric polypeptide of claim 19 further comprising (iii) a linker between the at least one single domain antibody and the carrier polypeptide.

24. (canceled)

25. The chimeric polypeptide of claim 19 having the amino acid sequence of SEQ ID NO: 16, 17 or 18, a variant of the amino acid sequence of SEQ ID NO: 16, 17 or 18 or a fragment of the amino acid sequence of SEQ ID NO: 16, 17 or 18.

26. (canceled)

27. The chimeric polypeptide of claim 19, wherein the chimeric polypeptide is-being associated with a toxic payload or a detectable label.

28. A nucleic acid molecule encoding the single domain antibody of claim 1.

29. The nucleic acid molecule according to claim 28 comprising at least one of SEQ ID NO: 21, 22, 23, 24, 25 or 26.

30. A vector comprising the nucleic acid molecule of claim 28.

31. A recombinant host cell comprising the nucleic acid molecule of claim 28.

32. A method of limiting the biological activity of carbonic anhydrase IX (CA-IX) expressed by a cell, the method comprising contacting the single domain antibody of claim 2 with CA-IX expressed by the cell so as to limit the biological activity of CA-IX in the cell when compared to a control cell contacted by a control single domain antibody that fails to specifically bind to the catalytic domain of CA-IX and reduce the biological activity of CA-IX, wherein the single domain antibody is capable of reducing the biological activity of CA-IX.

33. The method of claim 32 for the alleviation of a symptom of a cancer, the treatment of a cancer, the prevention of the re-occurrence of cancer, delivery of a toxic payload to the cell, and/or for improving the usefulness of a further therapeutic agent.

34. A method of detecting a cell expressing or overexpressing carbonic anhydrase IX (CA-IX), the method comprising:
(i) contacting the single domain antibody of claim 1 with the cell under conditions so as to allow the specific binding of the single domain antibody to the cell; and
(ii) determining the presence of a complex formed between the cell and the single domain antibody; and
(iii) detecting the cell as expressing or overexpressing CA-IX if the presence of the complex is determined to be present.

35-36. (canceled)


SEQUENCE LISTING (39 sequences)

SEQ ID NO: 1 - protein, 130 aa, Artificial sequence
  Amino acid sequence for sdCA9-1- VHH
QVQLVESGGGLVQAGGSLRLSCAASGFTFDDWAIGWFRQAPGKEREGVSCISKRHGTTHYADSVKGRFTISSDNAKNTVYLRMNGLKPEDTAVYYCAASSWGSCTVATMRDVDRYDYDYWGQGTQVTVSS

SEQ ID NO: 2 - protein, 8 aa, Artificial sequence
  Amino acid sequence of CDR1 of sdCA9-1- VHH
GFTFDDWA

SEQ ID NO: 3 - protein, 8 aa, Artificial sequence
  Amino acid sequence of CDR2 of sdCA9-1- VHH
ISKRHGTT

SEQ ID NO: 4 - protein, 23 aa, Artificial sequence
  Amino acid sequence of CDR3 of sdCA9-1- VHH
AASSWGSCTVATMRDVDRYDYDY

SEQ ID NO: 5 - protein, 133 aa, Artificial sequence
  Amino acid sequence for sdCA9-2-VHH
QVKLEESGGDLVQPGGSLRLSCAASGFTLDYYAIGWFRQAPGKEREGVSCFSSVDGSTYYADSVKGRFTISRDNAKNMVYLQMNSLKPEDTAIYYCAAELYGELGVATVQAMCRLTTPGGDYWGQGTQVTVSS

SEQ ID NO: 6 - protein, 8 aa, Artificial sequence
  Amino acid sequence of CDR1 of sdCA9-2-VHH
GFTLDYYA

SEQ ID NO: 7 - protein, 8 aa, Artificial sequence
  Amino acid sequence of CDR2 of sdCA9-2-VHH
FSSVDGST

SEQ ID NO: 8 - protein, 26 aa, Artificial sequence
  Amino acid sequence CDR3 of sdCA9-2-VHH
AAELYGELGVATVQAMCRLTTPGGDY

SEQ ID NO: 9 - protein, 118 aa, Artificial sequence
  A 118 amino acid sequence for CA9-3-VHH
QVQLVESGGGLVQAGDSLRLSCAASGGTFSRYGMGWFRQAPGKEREFVAAISGTGLNTYYMDSVKGRFTISRDNAENTVYLQMNSLKPEDTAVYYCARDRGGVEYDYWGQGTQVTVSS

SEQ ID NO: 10 - protein, 8 aa, Artificial sequence
  Amino acid sequence of CDR1 of sdCA9-3-VHH
GGTFSRYG

SEQ ID NO: 11 - protein, 8 aa, Artificial sequence
  Amino acid sequence of CDR2 of sdCA9-3-VHH
ISGTGLNT

SEQ ID NO: 12 - protein, 11 aa, Artificial sequence
  Amino acid sequence of CDR3 of sdCA9-3-VHH
ARDRGGVEYDY

SEQ ID NO: 13 - protein, 23 aa, Artificial sequence
  Leader amino acid sequence
EFATMEFGLSWVFLVAILKGVQC

SEQ ID NO: 14 - protein, 16 aa, Artificial sequence
  Linker amino acid sequence
AEPKSCDKTHTCPPCP

SEQ ID NO: 15 - protein, 216 aa, Artificial sequence
  hIgG1Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 385 aa, Artificial sequence
  Leader-sdCA9-1-VHH-Linker-hIgG1Fc
EFATMEFGLSWVFLVAILKGVQCQVQLVESGGGLVQAGGSLRLSCAASGFTFDDWAIGWFRQAPGKEREGVSCISKRHGTTHYADSVKGRFTISSDNAKNTVYLRMNGLKPEDTAVYYCAASSWGSCTVATMRDVDRYDYDYWGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 388 aa, Artificial sequence
  Leader-sdCA9-2-VHH-Linker-hIgG1Fc
EFATMEFGLSWVFLVAILKGVQCQVKLEESGGDLVQPGGSLRLSCAASGFTLDYYAIGWFRQAPGKEREGVSCFSSVDGSTYYADSVKGRFTISRDNAKNMVYLQMNSLKPEDTAIYYCAAELYGELGVATVQAMCRLTTPGGDYWGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 373 aa, Artificial sequence
  Leader-sdCA9-3-VHH-Linker-hIgG1Fc
EFATMEFGLSWVFLVAILKGVQCQVQLVESGGGLVQAGDSLRLSCAASGGTFSRYGMGWFRQAPGKEREFVAAISGTGLNTYYMDSVKGRFTISRDNAENTVYLQMNSLKPEDTAVYYCARDRGGVEYDYWGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - DNA, 38 bp, Artificial sequence
  Nucleic acid sequence of sense primer VHH Bbsl
tatgaagacaccaggcccaggtgcagctggtggagtct

SEQ ID NO: 20 - DNA, 31 bp, Artificial sequence
  Nucleic acid sequence of anti-sense primer VHH-BamHI
cgcgggatcctgaggagacggtgacctgggt

SEQ ID NO: 21 - DNA, 390 bp, Artificial sequence
  Nucleic acid sequence of sdCA9-1-VHH
caggtacagctggtggagtctgggggaggcttggtgcaggctggggggtctctgagactctcctgtgcagcctctggattcactttcgatgattgggccataggctggttccgccaggccccagggaaggagcgtgagggggtctcatgtattagtaagaggcatggtactacacactatgcagactccgtgaagggccgattcaccatctccagtgacaacgccaagaacacggtgtatctgcgaatgaacggcctgaaacctgaggacacggccgtttattactgtgcagcatcctcctggggatcatgtactgtagcgactatgagggacgttgatagatatgactatgactattggggccaggggacccaggtcaccgtctcctca

SEQ ID NO: 22 - DNA, 399 bp, Artificial sequence
  Nucleic acid sequence of sdCA9-2-VHH
caggtaaagctggaggagtctgggggagacttggtgcagcctggggggtctctgagactctcctgtgcagcctctggattcactttggattattatgccataggctggttccgccaggccccagggaaggagcgtgagggggtctcatgttttagtagtgttgatggtagcacatactatgcagactccgtgaagggccgattcaccatctccagagacaacgccaagaacatggtgtatctgcaaatgaacagcctgaaacctgaggacacagccatttattactgtgcagcagaattgtacggggagttgggagtggctactgttcaggctatgtgtcgcctgactactccggggggtgactactggggccaggggacccaggtcaccgtctcctca

SEQ ID NO: 23 - DNA, 354 bp, Artificial sequence
  Nucleic acid sequence of sdCA9-3-VHH
caggtgcagctggtggagtctgggggaggattggtgcaggctggggactctcttagactctcctgtgcagcctctggcggcaccttcagtaggtatggcatgggctggttccgccaggctccagggaaggagcgtgagtttgtcgcagctattagcgggactggtctgaatacatactatatggactccgtgaagggccgattcaccatctccagagacaacgccgagaacacggtgtatctgcaaatgaacagcctgaaacctgaggacacggccgtgtattactgtgccagagaccgaggaggcgttgagtatgactactggggccaggggacccaggtcaccgtctcctca

SEQ ID NO: 24 - DNA, 1095 bp, Artificial sequence
  Nucleic acid sequence of sdCA9-1-linker- hIgG1Fc
caggtacagctggtggagtctgggggaggcttggtgcaggctggggggtctctgagactctcctgtgcagcctctggattcactttcgatgattgggccataggctggttccgccaggccccagggaaggagcgtgagggggtctcatgtattagtaagaggcatggtactacacactatgcagactccgtgaagggccgattcaccatctccagtgacaacgccaagaacacggtgtatctgcgaatgaacggcctgaaacctgaggacacggccgtttattactgtgcagcatcctcctggggatcatgtactgtagcgactatgagggacgttgatagatatgactatgactattggggccaggggacccaggtcaccgtctcctcagctgaacccaagtcctgcgacaagacccacacctgtcccccctgccctgcccctgaactgctgggcggaccttccgtgttcctgttccccccaaagcctaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgcccccatcgaaaagaccatcagcaaggccaagggccagccccgcgagccccaggtgtacaccctgccccctagccgggacgagctgaccaagaatcaggtgtccctgacctgtctggtgaaaggcttctacccctccgatatcgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggctgaggatcc

SEQ ID NO: 25 - DNA, 1104 bp, Artificial sequence
  Nucleic acid sequence of sdCA9-2-linker- hIgG1Fc
caggtaaagctggaggagtctgggggagacttggtgcagcctggggggtctctgagactctcctgtgcagcctctggattcactttggattattatgccataggctggttccgccaggccccagggaaggagcgtgagggggtctcatgttttagtagtgttgatggtagcacatactatgcagactccgtgaagggccgattcaccatctccagagacaacgccaagaacatggtgtatctgcaaatgaacagcctgaaacctgaggacacagccatttattactgtgcagcagaattgtacggggagttgggagtggctactgttcaggctatgtgtcgcctgactactccggggggtgactactggggccaggggacccaggtcaccgtctcctcagctgaacccaagtcctgcgacaagacccacacctgtcccccctgccctgcccctgaactgctgggcggaccttccgtgttcctgttccccccaaagcctaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgcccccatcgaaaagaccatcagcaaggccaagggccagccccgcgagccccaggtgtacaccctgccccctagccgggacgagctgaccaagaatcaggtgtccctgacctgtctggtgaaaggcttctacccctccgatatcgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggctgaggatcc

SEQ ID NO: 26 - DNA, 1059 bp, Artificial sequence
  Nucleic acid sequence of sdCA9-3-linker- hIgG1Fc
caggtgcagctggtggagtctgggggaggattggtgcaggctggggactctcttagactctcctgtgcagcctctggcggcaccttcagtaggtatggcatgggctggttccgccaggctccagggaaggagcgtgagtttgtcgcagctattagcgggactggtctgaatacatactatatggactccgtgaagggccgattcaccatctccagagacaacgccgagaacacggtgtatctgcaaatgaacagcctgaaacctgaggacacggccgtgtattactgtgccagagaccgaggaggcgttgagtatgactactggggccaggggacccaggtcaccgtctcctcagctgaacccaagtcctgcgacaagacccacacctgtcccccctgccctgcccctgaactgctgggcggaccttccgtgttcctgttccccccaaagcctaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgcccccatcgaaaagaccatcagcaaggccaagggccagccccgcgagccccaggtgtacaccctgccccctagccgggacgagctgaccaagaatcaggtgtccctgacctgtctggtgaaaggcttctacccctccgatatcgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggctgaggatcc

SEQ ID NO: 27 - DNA, 657 bp, Artificial sequence
  Nucleic acid sequence  encoding hIgG1Fc (SEQ ID NO 15)
gcccctgaactgctgggcggaccttccgtgttcctgttccccccaaagcctaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgcccccatcgaaaagaccatcagcaaggccaagggccagccccgcgagccccaggtgtacaccctgccccctagccgggacgagctgaccaagaatcaggtgtccctgacctgtctggtgaaaggcttctacccctccgatatcgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggctgaggatcc

SEQ ID NO: 28 - DNA, 1675 bp, Artificial sequence
  Nucleic acid sequence  encoding CAR modular construct (SEQ ID NO 28)
  MISC_FEATURE (62)..(63): Insertion site for an antigen binding domain encoding sequence is between residues 62 and 63.
  MISC_FEATURE (62)..(63): Insertion site for an antigen binding domain encoding sequence is between nucleotides 62 and 63.
atgctcaggctgctcttggctctcaacttattcccttcaattcaagtaacaggagggtcttcgaagacttcctttgcgagacgacggtggcgggggatcaggtggtggaggtagcgggggagggggctcaggcggtacaactacgcctgcacctcgcccaccgaccccagcaccaaccatcgcttcacagcctttgagcctgcgaccagaggcatgtcgccctgctgcgggcggtgccgttcatactcgcggacttgattttgcgtgtgacgtcgtctcgccttctaagcccttttgggtgctggtggtggttggtggagtcctggcttgctatagcttgctagtaacagtggcctttattattttctgggtgaggaaacggggcagaaagaaactcctgtatatattcaaacaaccatttatgcgaccagtacaaactactcaagaggaagatggctgtagctgccgatttccagaagaagaagaaggaggatgtgaactgctgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgcgctagcgccacgaacttctctctgttaaagcaagcaggcgacgtggaagaaaaccccggtcccatggtgagcaagggcgaggaggacaacatggccagcctgcccgccacccacgagctgcacatcttcggcagcatcaacggcgtggacttcgacatggtgggccagggcaccggcaaccccaacgacggctacgaggagctgaacctgaagagcaccaagggcgacctgcagttcagcccctggattctggtgccccacatcggctacggcttccaccagtacctgccctaccccgacggcatgagccccttccaggccgccatggtggacggcagcggctaccaggtgcacaggaccatgcagttcgaggacggcgccagcctgaccgtgaactacaggtacacctacgagggcagccacatcaagggcgaggcccaggtgaagggcaccggcttccccgccgacggccccgtgatgaccaacagcctgaccgccgccgactggtgcaggagcaaaaagacctaccccaacgacaagaccatcatcagcaccttcaagtggagctacaccaccggcaacggcaagaggtacaggagcaccgccaggaccacctacaccttcgccaagcccatggccgccaactacctgaagaaccagcccatgtacgtgttcagaaagaccgagctgaagcacagcaagaccgagctgaacttcaaggagtggcagaaggccttcaccgacgtgatgggcatggacgagctgtacaagcccaagaagaagaggaaggtggaggacccccccgccgccaagagggtgaagctggactaa

SEQ ID NO: 29 - protein, 18 aa, Homo sapiens
MLRLLLALNLFPSIQVTG

SEQ ID NO: 30 - protein, 17 aa, Artificial sequence
  Synthetic Flexible Linker Domain (exemplary)
GGGGSGGGGSGGGGSGG

SEQ ID NO: 31 - protein, 45 aa, Homo sapiens
TTTPAPRPPTPAPTIASQPLSLRPEACRPAAGGAVHTRGLDFACD

SEQ ID NO: 32 - protein, 32 aa, Homo sapiens
PSKPFWVLVVVGGVLACYSLLVTVAFIIFWVR

SEQ ID NO: 33 - protein, 42 aa, Homo sapiens
KRGRKKLLYIFKQPFMRPVQTTQEEDGCSCRFPEEEEGGCEL

SEQ ID NO: 34 - protein, 114 aa, Homo sapiens
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 35 - protein, 5 aa, Artificial sequence
  Short flexible linker amino acid sequence
GGGGS

SEQ ID NO: 36 - DNA, 147 bp, Artificial sequence
  Bispecific T cell engager modular construct DNA sequence
  MISC_FEATURE (72)..(73): Insertion site for an antigen binding domain encoding sequence is between nucleotides 72 and 73
  MISC_FEATURE (99)..(100): Insertion site for an anti-CD3 variable heavy chain encoding sequence is between nucleotides 99 and 100
  MISC_FEATURE (144)..(145): Insertion site for an anti-CD3 variable light chain encoding sequence is between nucleotides 144 and 145
atggagtttgggctgagctgggttttcctcgttgctctttttagaggtgtccagtgtacaggagggtcttcggaagacttccttggaggaggcggaagtggcggtggtgggtcaggcggcggtgggagcggaggaggtggaagctag

SEQ ID NO: 37 - protein, 389 aa, Artificial sequence
  sdCA9-1-VHH CAR amino acid sequence, exemplary sequence for a single binder CAR construct
MLRLLLALNLFPSIQVTGQVQLVESGGGLVQAGGSLRLSCAASGFTFDDWAIGWFRQAPGKEREGVSCISKRHGTTHYADSVKGRFTISSDNAKNTVYLRMNGLKPEDTAVYYCAASSWGSCTVATMRDVDRYDYDYWGQGTQVTVSSPSGGGGPSTTTPAPRPPTPAPTIASQPLSLRPEACRPAAGGAVHTRGLDFACDPSKPFWVLVVVGGVLACYSLLVTVAFIIFWVRKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - protein, 392 aa, Artificial sequence
  sdCA9-2-VHH CAR amino acid sequence
MLRLLLALNLFPSIQVTGQVKLEESGGDLVQPGGSLRLSCAASGFTLDYYAIGWFRQAPGKEREGVSCFSSVDGSTYYADSVKGRFTISRDNAKNMVYLQMNSLKPEDTAIYYCAAELYGELGVATVQAMCRLTTPGGDYWGQGTQVTVSSPSGGGGPSTTTPAPRPPTPAPTIASQPLSLRPEACRPAAGGAVHTRGLDFACDPSKPFWVLVVVGGVLACYSLLVTVAFIIFWVRKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 39 - protein, 222 aa, Artificial sequence
  sdCAIX2-CD3scFv amino acid sequence, exemplary sequence for a CAIX bispecific immune engager construct
  SITE (216)..(217): Insertion site for a CD3-specific scFv sequence is between residues 216 and 217
MEFGLSWVFLVALFRGVQCTGQVKLEESGGDLVQPGGSLRLSCAASGFTLDYYAIGWFRQAPGKEREGVSCFSSVDGSTYYADSVKGRFTISRDNAKNMVYLQMNSLKPEDTAIYYCAAELYGELGVATVQAMCRLTTPGGDYWGQGTQVTVSSGGGGSGGGGSGGGGSGGTTTPAPRPPTPAPTIASQPLSLRPEACRPAAGGAVHTRGLDFACDHHHHHH